(19) United States
(12) Patent Application Publication
(10) Pub. No.: US 2020/0109446 A1
(43) Pub. Date: Apr. 9, 2020

(54) CHIP HYBRIDIZED ASSOCIATION-MAPPING PLATFORM AND METHODS OF USE

(71) Applicant: BOARD OF REGENTS, THE UNIVERSITY OF TEXAS SYSTEM, Austin, TX (US)

(72) Inventors: Ilya FINKELSTEIN, Austin, TX (US); Cheulhee JUNG, Austin, TX (US); John HAWKINS, Austin, TX (US); Stephen Knox JONES, Manor, TX (US); James RYBARSKI, Austin, TX (US); Fatema A. SAIFUDDIN, Austin, TX (US); Cagri SAVRAN, West Lafayette, IN (US); Andrew D. ELLINGTON, Austin, TX (US); William H. PRESS, Austin, TX (US)

(21) Appl. No.: 16/622,441

(22) PCT Filed: Jun. 14, 2018
(86) PCT No.: PCT/US2018/037493

§ 371 Date: Dec. 13, 2019

Related U.S. Application Data
(60) Provisional application No. 62/519,502, filed on Jun. 14, 2017.

(51) Int. Cl. C12Q 1/6837; G01N 21/64; C12Q 1/6874; C40B 40/06; C40B 30/04; G01N 33/53; G01N 33/543; G16B 40/10; G16B 20/30

(52) U.S. Cl. CPC C12Q 1/6837; G01N 21/6428; C12Q 1/6874; C40B 40/06; C40B 30/04; G01N 33/5308; C12Q 2563/107; G16B 40/10; G16B 20/30; G01N 2021/6439; G01N 2500/04; C12Q 2522/101; G01N 33/54386

(57) ABSTRACT

Disclosed herein is a method and system for a high-throughput, quantitative analysis of protein-DNA interactions on synthetic and genomic DNA. This system and method makes use of sequencing chips which have already been used to carry out sequencing and is therefore environmentally friendly, as well as efficient and accurate.

### CROSS-REFERENCE TO RELATED APPLICATIONS

This application claims benefit of U.S. Provisional Application No. 62/519,502, filed Jun. 14, 2017, incorporated herein by reference in its entirety.

### GOVERNMENT SUPPORT CLAUSE

This invention was made with government support under Grant No. 1453358 awarded by the National Science Foundation and Grant No. ACG53051 awarded by the National Institutes of Health. The government has certain rights in the invention.

### BACKGROUND

The interaction between proteins and nucleic acids plays a fundamental role in virtually every cellular event, particularly in gene regulation and nucleic acid replication. However, the interactions between proteins and nucleic acids are not well understood or easily predicted. Different methods have been used to study these interactions. For example, binding small ligands with DNA has been studied by several well-characterized techniques, such as protection of nucleic acids in a complex against chemical modifications, nuclease footprinting assays, separation of the complexes by electrophoresis, dialysis and optical methods in the case of small ligands.

Immobilization of oligonucleotides on filters or glass surfaces also provides a means to assay protein-DNA interactions. All of these methods are usually applied to discriminate stringent specific binding from nonspecific binding, and these findings usually require painstaking research in order to determine the nucleic acid sequence for which the protein has the highest specificity and/or affinity. Nucleic acid binding proteins have been discovered that interact only with single-stranded (ss) DNA or double-stranded (ds)DNA, ssRNA, or dsRNA and these proteins often have different degrees of DNA or RNA sequence specificity. To date, there has not been a large-scale, high-throughput chip for determining protein-nucleic acid binding sequence. Nor is there a method for applying advanced imaging modalities (i.e., Förster resonance energy transfer, FRET) to high-throughput on-chip protein-nucleic acid interactions. Thus, there continues to be a need to readily characterize the interactions between nucleic acids and proteins.

### SUMMARY

Disclosed herein is a method for determining protein-nucleic acid interactions, the method comprising: exposing nucleic acid clusters on a high-throughput array to one or more fluorescently labeled proteins; and detecting protein-nucleic acid interactions by fluorescent imaging.

Also disclosed herein is a chip hybridized association-mapping platform for determining protein-nucleic acid interaction, the platform comprising nucleic acid clusters on a high-throughput array and one or more fluorescently labeled proteins.

### BRIEF DESCRIPTION OF THE DRAWINGS

The accompanying drawings, which are incorporated in and constitute a part of this specification, illustrate several embodiments and together with the description illustrate the disclosed compositions and methods.

FIGS. 1A, 1B, 1C, 1D, 1E, 1F, 1G, and 1H show a chip-hybridized affinity-mapping platform (CHAMP). FIG. 1A shows an overview of the CHAMP workflow. DNA is regenerated on a sequenced NGS chip. A subset of clusters is hybridized to fluorescent oligonucleotides (alignment markers, magenta). Fluorescent proteins are incubated in the chip (green) and the fluorescent intensities at each DNA cluster are recorded via total internal reflection fluorescence (TIRF) microscopy. A computational pipeline uses the alignment markers to identify the DNA sequences of all fluorescent clusters. FIG. 1B shows a schematic representation of the T. fusca Cascade protein complex. Cse1 is shown in purple, Cas7 subunits are shown in alternating blue and yellow, and all other subunits are collectively represented in gray. The target DNA is gray, the protospacer adjacent motif (PAM) and seed regions are black, while the crRNA is red. FIG. 1C shows that increasing concentrations of fluorescent Cascade complexes are incubated in the regenerated NGS chip and (FIG. 1D) the apparent binding affinities for each DNA sequence are obtained by fitting the fluorescent intensities to the Hill equation. The lowest-affinity curve in (black dashed line, D) reports non-specific binding of Cascade to off-target DNA clusters. FIG. 1E shows an illustration of the synthetic oligonucleotide library used for CHAMP. FIG. 1F shows an overview of the randomized library used for these studies. The bar graph represents the number of unique sequences used in the CHAMP experiments with increasing substitutions from the ideal PAM and protospacer sequence. The bars are shaded to indicate the percent coverage of the relevant sequence space. Violin plots indicate the number of DNA clusters observed per sequence in the CHAMP dataset. Only sequences represented by five or more unique DNA clusters are included in the analysis (dashed line). FIG. 1G shows that CHAMP experiments were highly repeatable between two independently sequenced NGS chips. The gray zones indicate ABAs that fell outside of the experimentally defined cutoff for non-specific binding. The r-value was calculated omitting gray zones. FIG. 1H shows a rank-ordered list of all 35,968 ABAs that were measured via CHAMP. The gray line represents the standard deviation as measured by bootstrap analysis. See also FIG. 2-5.

FIGS. 2A, 2B, and 2C show an overview of the CHAMP experimental platform, Related to FIG. 1. FIG. 2A shows that MiSeq chips are imaged via prism-based TIRF microscopy on a custom-built microscope stage. Three lasers are used to excite the fluorophores. Exposure times are controlled by three computer-controlled shutters (S1-S3). Neutral density filters (F1-F3) are used to control the laser intensity, long-pass dichroic mirrors (DM1-DM2) combine the laser beams into a single path, mirrors (M1-M2) direct the beams through a prism to generate an evanescent excitation field for TIRF imaging. The reflected beams are blocked at a beam stop (BS). The emitted photons pass through the objective and a computer-controlled filter wheel (FW) that removes residual laser excitation. A dichroic mirror (DM3) separates spectrally distinct fluorophore emissions, which are directed towards two electron-multiplying charge coupled device cameras (EM-CCDs) for wide-field imaging. Reagents are delivered to the microfluidic chip via a computer-controlled syringe pump. Temperature is controlled via a custom-built controller. FIG. 2B shows a diagram of the MiSeq chip adapter. The MiSeq chip is inserted into the chip holder and secured to the base plate in combination with the tubing holder. Microfluidic tubing is fit into the tubing holder, passed between the tubing guide and pressure plate, and mated with the MiSeq chip. FIG. 2C shows the regenerating DNA clusters on a sequenced MiSeq chip. After sequencing, the chip contains residual fluorescence in all emission channels (left). The residual fluorescence and sequenced DNA strands are chemically stripped and the DNA is regenerated (middle two panels). PhiX clusters are labeled with a fluorescent oligonucleotide (magenta) for downstream image alignment. Cascade is incubated in the chip and binds a subset of the DNA clusters. Cascade can be visualized after the addition of fluorescent anti-FLAG antibody, (fifth panel, green). After chip regeneration, all fluorescent signals are sensitive to DNAse I treatment, indicating that these signals originate from DNA clusters.

FIGS. 3A, 3B, 3C, 3D, and 3E show cluster identification and linear discriminant analysis (LDA), Related to FIG. 1. FIG. 3A shows a flow chart for cluster identification. FIG. 3B shows a representative alignment. The first image (green) shows the alignment marker coordinates, each represented by a radially symmetric Gaussian. These coordinates are found by mapping all reads against the PhiX genome, and aligning the mapped reads with a TIRF microscope image with fluorophores attached to all alignment markers (magenta, middle). The third image shows the overlap of the synthetic and experimental images (overlap seen as white). FIG. 3C shows an example 7×7 pixel images centered on aligned FASTQ points for targeted and non-targeted clusters. FIG. 3D shows linear discriminant analysis (LDA) was used to train pixel weights using sub-images as in (C) from sequences known to be on or off. Shown are the trained weights. 7×7 pixels sub-imaged were found to be optimal. To calculate intensity scores for Kd calculations, these weights, with negative values set to zero, are multiplied by the corresponding pixel values and summed. FIG. 3E shows the ROC (receiver operating characteristic) curve using LDA scores from (D) for classification of a test set of approximately 75,000 points. Perfect target A sequences were used as ground-truth positive values, and non-target sequences as ground-truth negative values when calculating the true- and false-positive rates (TPR, FPR). The extremely high area under the curve (AUC) of 0.999 indicates both very good alignment of the sequence coordinates and microscope images, as well as high fidelity of the chemistry in illuminating the correct clusters and only the correct clusters.

FIG. 4A shows fluorescent signal intensity remains constant throughout the CHAMP experiment. Cascade (10 nM) was incubated on an NGS chip for 10 minutes at 60° C., then washed and labeled with anti-FLAG Alexa488 antibody. Images were then collected every five minutes for one hour. The graph above represents the mean intensity of all clusters containing the perfectly basepaired target DNA sequence. Error bars: S.E.M. The normalized data was fit to an exponential decay curve to estimate the half-life (dashed line).

FIG. 4B shows the estimating the error in the ABA. Bootstrap ABA values were calculated for the perfect target sequence with all numbers of clusters between 3 and 100. Shown are the average errors (blue points) and 90% confidence intervals of error (red points), using the ABA fit with 2,000 clusters as reference. The gray dotted line shows a cutoff of 5 clusters, with average ABA error of approximately 0.2 kBT. Solid lines indicate a fit to the data.

FIG. 4C shows sequencing quality. Information from both paired-end reads was used to produce high confidence inferred sequences. A simple Bayesian model was developed for inferring each base, assuming independent errors in each position and a flat prior. For each position, this gives:

P(tᵢ=b|R₁ᵢ,Q₁ᵢ,R₂ᵢ,Q₂ᵢ)αP(R₁ᵢ,|tᵢ=b,Q₁ᵢ,)·P(R₂ᵢ,|tᵢ=b,Q₂ᵢ,)

where i is the position in the aligned sequence, ti is the true sequence base, b is a base identity (A, C, G, or T), R1i and R2i are the read bases, and Q1i and Q2i are the Phred scores. Maximum a posteriori (MAP) values were taken as the inferred sequence. Shown above are all values for P(R=r|t=b, Q) observed from 10 billion read bases in PhiX reads mapped without gaps to the Illumina PhiX genome, observed to have the following mutations relative to the NCBI PhiX genome gi|9626372: G587A, G833A, A2731G, C2811T, C3133T. The gray dashed line shows the implied probability for each mismatch given the Phred score, and was used wherever observed values were not available. Base reads other than A, C, G, or T and bases with Phred scores less than or equal to 2, which Illumina reserves for special use, were discarded as missing data.

FIGS. 5A, 5B, 5C, 5D, 5E, 5F, and 5G show comprehensive profiling of Cascade-DNA interactions. FIG. 5A shows the change in ABA for all 105 possible single-base substitutions along the minimal PAM and the target DNA. Negative values indicate a reduced ABA relative to the best PAM and perfectly paired DNA target. Error bars: S.D. obtained via bootstrapping. FIG. 5B shows that CHAMP profiling was performed on two distinct DNA libraries (blue and red dots). The resulting data was used to construct a minimal binding model shown in (C) and (D) that accurately describes the data obtained from both CHAMP datasets. FIG. 5C shows the position-dependent substitution penalties and (FIG. 5D) position-independent nucleotide preferences obtained from the binding model. FIG. 5E shows the change in ABA for all dinucleotide substitutions. The triangular matrix represents the average of CHAMP measurements acquired on two independent chips. The PAM is in the upper left-hand corner. Gray regions indicate insufficient data. As an example, the inset shows an enlarged 3×3 dinucleotide substitution matrix showing all possible substitutions for positions A₁₂ and C₉. FIG. 5F shows a schematic representation of T. fusca Cascade highlighting contribution of PAM positions −1 to −6, and the three-nucleotide periodicity. FIG. 5G shows models representing the three nucleotide periodicity imposed by the protruding Cas7 finger (residues 193-211) (top) and steric clash with adjacent amino acids (R19, M173, D183 and K271; transparent DNA for clarity) (bottom) based on E. coli Cascade.

FIGS. 6A, 6B, 6C, and 6D show profiling off-target Cascade binding in a human exome. FIG. 6A shows the CHAMP-Exome analysis pipeline. Human genomic DNA is randomly sheared and enriched for exome sequences (blue) using standard oligonucleotide hybridization and bead pull-down protocols. After enrichment and adapter ligation, the exome is sequenced on a MiSeq chip, which is then used for CHAMP. Apparent Binding Affinities (ABAs) at each position in the exome were measured via CHAMP. FIG. 6B shows the maximum ABA values in each gene, ordered by rank. The dashed line indicates ABAs that fell outside of the experimentally defined cutoff for non-specific binding. Inset: histogram of genes that show measurable off-target binding. The gray zone indicates genes that had ABAs greater than 3 k_{B}T. Red dots in (B) indicate three representative genes with strong off-target binding sites, further described in (C). FIG. 6C shows an example high-affinity peaks. ABA is measured at each position in each gene using all reads overlapping that position. A high-affinity site thus appears as a peak in ABA whose width is a function of the DNA shearing length distribution. Shown are the measured ABAs at each position in a few genes containing high-ABA peaks. The ABAs spanning each gene are shown in blue (left y-axis) and the sequencing coverage in purple (right y-axis). Exon boundaries are shown as the minor ticks along the x-axis, and cause sharp changes in displayed ABA and coverage values. FIG. 6D shows sequence logo generated from a 210-bp window centered around each of the ABA peaks >3 k_{B}T. Image generated with WebLogo.

FIGS. 7A and 7B show the exome sequence length distribution and expected peak shape, Related to FIG. 6. FIG. 7A shows the distribution of exome sequence lengths. The DNA was sheared and sized to a nominal DNA fragment length of approximately 150 bp. The observed mean DNA length and coefficient of variation were 170 bp and 22%, respectively. FIG. 7B shows the resolution of measuring a DNA binding site in a randomly sheared DNA sample depends on the fragment length distribution and the coverage depth of each fragment. The shear lengths from (A) were used to calculate the probability that a random read covering a nearby base would also cover a target binding site (red dashed curve, see Methods). In the limit of infinite coverage and perfectly random shearing, this gives the range of influence a binding site has on measurements for nearby bases, and hence provides an estimate for the resolution of this method. In the current experiment, the full width at half maximum (FWHM) of this peak is 162 bp. The observed resolution was calculated by normalizing and averaging the thirty highest-affinity binding peaks (blue curve). The experimentally observed FWHM was 210 bp and was used to define the resolution for this experiment. Deviations from the expected peak shape (red) are due to finite coverage, bias in shearing sites, and the non-linear map from reads included to measure ABA.

FIGS. 8A, 8B, 8C, and 8D shows three-color CHAMP reveals DNA sequence-dependent Cas3 recruitment. FIG. 8A shows an experimental strategy overview. Fluorescent Cascade is first incubated in the regenerated chips. Next, fluorescent Cas3 is introduced into the same chip. FIG. 8B shows that most DNA-bound Cascade complexes readily bind Cas3 (white arrow, right inset). However, a small subset of clusters shows reduced Cas3 binding (green arrow, right insert). FIG. 8C shows an analysis of the fluorescent Cascade and Cas3 intensities at all sequences with a single nucleotide mismatch. Points below the diagonal indicate reduced Cas3 binding. Color bar indicates the position of the mismatch and the labels indicate the identity of the substituted bases. The gray point is a negative control indicating the background fluorescent intensity, as measured at non-specific DNA sequences on the same chip. Error bars: SEM of at least 213 independent clusters. FIG. 8D shows an analysis of the position-dependent Cas3 recruitment penalties. The solid line is an average of the three possible substitutions

FIGS. 9A, 9B, 9C, 9D, 9E, 9F, and 9G show repurposing MiSeq chips for FRET-CHAMP and adapting CHAMP for Illumina HiSeq sequencers. FIG. 9A shows a subset of DNA clusters on a MiSeq chip were hybridized with an oligonucleotide containing either a Cy3 dye (top), or a Cy3 and Cy5 dyes separated by 16 nucleotides (bottom). FIG. 9B shows that Cy3 was illuminated with a 532 nm laser (15 mW intensity at the prism face) and fluorescent images were simultaneously collected in both the Cy3 and Cy5 channels. FIG. 9C shows the mean FRET efficiency from at least 100 clusters computed from five different fields-of-view. Error-bars: S.D. FIG. 9D shows a photograph of a HiSeq microfluidic chip. The HiSeq chip has eight separate lanes. The HiSeq 4000 was used, which typically generates ˜1-5 billion unique DNA clusters per chip. FIG. 9E shows a subset of fluorescent PhiX clusters imaged in a 0.26×0.87 mm region of the fourth lane using TIRF microscopy. This composite image is assembled from eight partially overlapping fields-of-view. The CHAMP image analysis pipeline was used to identify these clusters in the corresponding HiSeq sequencing (FASTQ) file. FIG. 9F shows an expanded view of the PhiX clusters (magenta), the aligned FASTQ coordinates image (green), and the merged image of the two (right). The aligned FASTQ coordinates are depicted as Gaussian convolutions to mimic the diffraction-limited fluorescent spots seen in TIRF microscopy. FIG. 9G shows a maximum cross-correlation of the TIRF image in (F) with HiSeq FASTQ tiles shows strong signal for correct alignment. Maximum cross-correlation was calculated for FASTQ tiles that neighbor the region imaged in (E). Maximum correlation of the TIRF image with incorrect FASTQ tiles is primarily a function of the density of the alignment markers and size of the tiles, and therefore relatively constant for tiles in the same lane. The signal-to-noise ratio (SNR) of the correct alignment in the correct tile (shown in red) is nearly 3, well above the relatively conservative SNR threshold of 1.4 (shown as grey background). The background noise level (SNR=1) was determined by using the maximum cross correlation value of tiles in the same lane known not to contain the image (E).

### DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. Definitions

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about.” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Numeric ranges are inclusive of the numbers defining the range. The term about is used herein to mean plus or minus ten percent (10%) of a value. For example, “about 100” refers to any number between 90 and 110.

The term “library” herein refers to a collection or plurality of template molecules, i.e., target DNA duplexes, which share common sequences at their 5′ ends and common sequences at their 3′ ends. Use of the term “library” to refer to a collection or plurality of template molecules should not be taken to imply that the templates making up the library are derived from a particular source, or that the “library” has a particular composition. By way of example, use of the term “library” should not be taken to imply that the individual templates within the library must be of different nucleotide sequence or that the templates must be related in terms of sequence and/or source.

The term “Next Generation Sequencing (NGS)” herein refers to sequencing methods that allow for massively parallel sequencing of clonally amplified and of single nucleic acid molecules during which a plurality, e.g., millions, of nucleic acid fragments from a single sample or from multiple different samples are sequenced in unison. Non-limiting examples of NGS include sequencing-by-synthesis, sequencing-by-ligation, real-time sequencing, and nanopore sequencing.

The term “base pair” or “bp” as used herein refers to a partnership (i.e., hydrogen bonded pairing) of adenine (A) with thymine (T), or of cytosine (C) with guanine (G) in a double stranded DNA molecule. In some embodiments, a base pair may comprise A paired with Uracil (U), for example, in a DNA/RNA duplex.

The term “complementary” herein refers to the broad concept of sequence complementarity in duplex regions of a single polynucleotide strand or between two polynucleotide strands between pairs of nucleotides through base-pairing. It is known that an adenine nucleotide is capable of forming specific hydrogen bonds (“base pairing”) with a nucleotide, which is thymine or uracil. Similarly, it is known that a cytosine nucleotide is capable of base pairing with a guanine nucleotide.

The term “essentially complementary” herein refers to sequence complementarity in duplex regions of a single polynucleotide strand or between two polynucleotide strands of an adaptor wherein the complementarity is less than 100% but is greater than 90%, and retains the stability of the duplex region under conditions for covalent linking of the adaptor to a target DNA duplex.

The term “purified” herein refers to a molecule is present in a sample at a concentration of at least 90% by weight, or at least 95% by weight, or at least 98% by weight of the sample in which it is contained.

The term “isolated” herein refers to a nucleic acid molecule that is separated from at least one other molecule with which it is ordinarily associated, for example, in its natural environment. An isolated nucleic acid molecule includes a nucleic acid molecule contained in cells that ordinarily express the nucleic acid molecule, e.g., via chromosomal expression, but the nucleic acid molecule is present extrachromosomally or at a chromosomal location that is different from its natural chromosomal location.

The term “nucleotide” herein refers to a monomeric unit of DNA or RNA consisting of a sugar moiety (pentose), a phosphate, and a nitrogenous heterocyclic base. The base is linked to the sugar moiety via the glycosidic carbon (1′ carbon of the pentose) and that combination of base and sugar is a nucleoside. When the nucleoside contains a phosphate group bonded to the 3′ or 5′ position of the pentose it is referred to as a nucleotide. A sequence of polymeric operatively linked nucleotides is typically referred to herein as a “base sequence.” “nucleotide sequence,” or nucleic acid or polynucleotide “strand,” and is represented herein by a formula whose left to right orientation is in the conventional direction of 5′-terminus to 3′-terminus, referring to the terminal 5′ phosphate group and the terminal 3′ hydroxyl group at the “5′” and “3′” ends of the polymeric sequence, respectively.

The terms “oligonucleotide”, “polynucleotide” and “nucleic acid” herein refer to a molecule including two or more deoxyribonucleotides and/or ribonucleotides, preferably more than three. Its exact size will depend on many factors, which in turn depend on the ultimate function or use of the oligonucleotide. The oligonucleotide may be derived synthetically or by cloning or from a natural (e.g., genomic) source. As used herein, the term “polynucleotide” refers to a polymer molecule composed of nucleotide monomers covalently bonded in a chain. DNA (deoxyribonucleic acid) and RNA (ribonucleic acid) are examples of polynucleotides.

“Optional” or “optionally” means that the subsequently described event or circumstance may or may not occur, and that the description includes instances where said event or circumstance occurs and instances where it does not.

As used herein, “nucleic acid sequencing data”, “nucleic acid sequencing information”, “nucleic acid sequence”, “genomic sequence”, “genetic sequence”, “fragment sequence”, or “nucleic acid sequencing read” denotes any information or data that is indicative of the order of the nucleotide bases (e.g., adenine, guanine, cytosine, and thymine/uracil) in a molecule (e.g., a whole genome, a whole transcriptome, an exome, oligonucleotide, polynucleotide, fragment, etc.) of DNA or RNA.

Reference to a base, a nucleotide, or to another molecule may be in the singular or plural. That is, “a base” may refer to a single molecule of that base or to a plurality of the base, e.g., in a solution.

As used herein, the term “target nucleic acid” or “target nucleotide sequence” refers to any nucleotide sequence (e.g., RNA or DNA), the manipulation of which may be deemed desirable for any reason by one of ordinary skill in the art, including protein interaction. In some contexts, “target nucleic acid” refers to a nucleotide sequence whose nucleotide sequence is to be determined or is desired to be determined. In some contexts, the term “target nucleotide sequence” refers to a sequence to which an interaction with a protein is to be determined.

As used herein, the term “region of interest” refers to a nucleic acid or protein that is analyzed (e.g., using one of the compositions, systems, or methods described herein). In some embodiments, the region of interest is a portion of a genome or region of genomic DNA (e.g., comprising one or chromosomes or one or more genes). In some embodiments, mRNA expressed from a region of interest is analyzed.

As used herein, the term “corresponds to” or “corresponding” is used in reference to a contiguous nucleic acid or nucleotide sequence (e.g., a subsequence) that is complementary to, and thus “corresponds to”, all or a portion of a target nucleic acid sequence.

The phrase “sequencing run” refers to any step or portion of a sequencing experiment performed to determine some information relating to at least one biomolecule (e.g., nucleic acid molecule).

As used herein, “complementary” generally refers to specific nucleotide duplexing to form canonical Watson-Crick base pairs, as is understood by those skilled in the art. However, complementary also includes base-pairing of nucleotide analogs that are capable of universal base-pairing with A, T, G or C nucleotides and locked nucleic acids that enhance the thermal stability of duplexes. One skilled in the art will recognize that hybridization stringency is a determinant in the degree of match or mismatch in the duplex formed by hybridization.

The term “protein” refers to a large molecule comprising one or more chains of amino acids. The protein may further comprise of components made up of nucleotides. The protein may be negatively charged or positively charged. The protein may have a vast array of functions, including but not limited to, catalysis, gene regulation, responding to stimuli and the like.

The term “peptide” refers to a small molecule comprising one or more amino acids. The peptide may be negatively or positively charged.

The terms “artificial protein” and “synthetic protein” may be used interchangeably, and refer to man-made molecules that mimic the function and structure of naturally occurring proteins. An artificial protein may have genetic sequences that are not seen in naturally occurring proteins. An artificial protein may bind to specific recognition sequences.

The term “recognition sequence” refers to a nucleic acid sequence or subset thereof, to which the nucleic-acid binding domain motif of a protein is specific to. That is, the recognition sequence is a nucleic acid sequence that a protein has specificity for. A particular protein may have specificity for a particular nucleic acid sequence, which is the recognition sequence for that particular protein.

The term “enhance” in reference to fluorescence for the purposes of this disclosure, refers to any process that increases the fluorescence intensity of a given substance. Enhancement may be a result of, but not limited to, excited state reactions, energy transfer, electron transfer, complex formation, colloidal quenching and the like. Enhancement may be static or dynamic. The term “enhanceable” should be construed accordingly.

The term “quench” in reference to fluorescence for the purposes of this disclosure, refers to any process that decreases the fluorescence intensity of a given substance. Quenching may be a result of, but not limited to, excited state reactions, energy transfer, electron transfer, complex formation, colloidal quenching and the like. Quenching may be static or dynamic. The term “quenchable” should be construed accordingly.

The terms “restore” and “recover” in reference to fluorescence for the purposes of this disclosure, may be used interchangeably, and refer to the increase in fluorescence following initial quenching. The terms “restoration” and “recovery” should be construed accordingly.

As used herein, a “system” denotes a set of components, real or abstract, comprising a whole where each component interacts with or is related to at least one other component within the whole.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. Methods and Platforms

Disclosed herein is chip hybridized association-mapping platform (CHAMP): a method for determining protein-nucleic acid interactions, the method comprising: exposing nucleic acid clusters on a high-throughput array to one or more fluorescently labeled proteins: and detecting protein-nucleic acid interactions by fluorescent imaging. CHAMP adds to a growing toolbox of high-throughput methods for determining aspects of protein-DNA interactions. CHAMP offers three key advantages over previous approaches. First, using a conventional fluorescence microscope opens new experimental configurations, including multi-color co-localization and time-dependent kinetic experiments. The excitation and emission optics can also be readily adapted for FRET, and other advanced imaging modalities. Second, complete fluidic access to the chip allows addition of other protein components during a biochemical reaction. Third, the computational strategy for aligning sequencer outputs to fluorescent datasets is applicable to all modern Illumina® sequencers, including the MiSeq™, NextSeq™, and HiSeq™ platforms.

The CHAMP methods and platform disclosed herein can be broadly classified by the information content (from hundreds to millions of unique interactions probed in parallel), the types of DNA sequences that can be interrogated (e.g., synthetic oligonucleotides and/or genomic libraries), and the detection schemes used to infer biophysical parameters. CHAMP differs from most of other high-throughput methods because all profiling experiments are carried out on sequencing chips, which may have already been used in sequencing reaction, such as an Illumina® chip, which can be generated during the Illumina®-based next generation DNA sequencing workflow. For example, current MiSeq™ chips generate up to 25 million unique DNA clusters, and the HiSeq™ generates up to 10 billion unique DNA clusters, and both are compatible with synthetic and genomic DNA libraries. Proteins are fluorescently labeled and a conventional fluorescence microscope is used to image protein binding to each DNA cluster. Using a fluorescence microscope opens new experimental configurations, including multi-color co-localization, time-dependent kinetic experiments, FRET, and other advanced imaging modalities.

a) Nucleic Acids/Sequencing

The individual target nucleic acid molecule (also referred to herein as a “nucleic acid cluster” when in a cluster arrangement, as discussed herein) may be any nucleic acid amenable to nucleotide sequence analysis and protein interaction detection. The target nucleic acid may be a DNA or an RNA molecule, either natural-occurring material or synthesized. The target nucleic acid molecule may be isolated, purified or partially purified. The target nucleic acid molecule may be derived from a tissue, a cell or a body fluid (such as, but not limited to, blood, plasma or saliva), or a fraction thereof (e.g., a nuclear fraction). The target nucleic acid may be in a liquid solution (e.g., a suitable buffer solution) or a solid matrix (e.g., a gel matrix such as an acrylamide gel or an agarose gel). Methods of the present disclosure may preferably include a step of isolating a target nucleic acid. The nucleic acid may have been previously sequenced, and attached to a chip.

In some embodiments, immobilized DNA fragments are amplified using cluster amplification methodologies as exemplified by the disclosures of U.S. Pat. Nos. 7,985,565 and 7,115,400, the contents of each of which is incorporated herein by reference in its entirety. The incorporated materials of U.S. Pat. Nos. 7,985,565 and 7,115,400 describe methods of solid-phase nucleic acid amplification which allow amplification products to be immobilized on a solid support in order to form arrays comprised of clusters or “colonies” of immobilized nucleic acid molecules. Each cluster or colony on such an array is formed from a plurality of identical immobilized polynucleotide strands and a plurality of identical immobilized complementary polynucleotide strands. The arrays so-formed are generally referred to herein as “clustered arrays”. The products of solid-phase amplification reactions such as those described in U.S. Pat. Nos. 7,985,565 and 7,115,400 are so-called “bridged” structures formed by annealing of pairs of immobilized polynucleotide strands and immobilized complementary strands, both strands being immobilized on the solid support at the 5′ end, preferably via a covalent attachment. Cluster amplification methodologies are examples of methods wherein an immobilized nucleic acid template is used to produce immobilized amplicons. Other suitable methodologies can also be used to produce immobilized amplicons from immobilized DNA fragments produced according to the methods provided herein. For example one or more clusters or colonies can be formed via solid-phase PCR whether one or both primers of each pair of amplification primers are immobilized. These clusters can then be used to determine nucleic acid-protein interactions.

In some embodiments of the technology, nucleic acid sequence data are generated prior to determination of protein interaction using CHAMP with the nucleic acid target. Various embodiments of nucleic acid sequencing platforms (e.g., a nucleic acid sequencer) include components as described herein and elsewhere in the art. For example, a sequencing instrument can include a fluidic delivery and control unit, a sample processing unit, a signal detection unit, and a data acquisition, analysis and control unit. Various embodiments of the instrument provide for automated sequencing that is used to gather sequence information from a plurality of sequences in parallel and/or substantially simultaneously.

In some embodiments, the sample processing unit includes a sample chamber, such as flow cell, a substrate, a micro-array, a multi-well tray, or the like. The sample processing unit can include multiple lanes, multiple channels, multiple wells, or other means of processing multiple sample sets substantially simultaneously. Additionally, the sample processing unit can include multiple sample chambers to enable processing of multiple runs simultaneously. In particular embodiments, the system can perform signal detection on one sample chamber while substantially simultaneously processing another sample chamber. Additionally, the sample processing unit can include an automation system for moving or manipulating the sample chamber. In some embodiments, the signal detection unit can include an imaging or detection sensor. For example, the imaging or detection sensor (e.g., a fluorescence detector or an electrical detector) can include a CCD, a CMOS, an ion sensor, such as an ion sensitive layer overlying a CMOS, a current detector, or the like. The signal detection unit can include an excitation system to cause a probe, such as a fluorescent dye, to emit a signal. The detection system can include an illumination source, such as arc lamp, a laser, a light emitting diode (LED), or the like. In particular embodiments, the signal detection unit includes optics for the transmission of light from an illumination source to the sample or from the sample to the imaging or detection sensor. Alternatively, the signal detection unit may not include an illumination source, such as for example, when a signal is produced spontaneously as a result of a sequencing reaction. For example, a signal can be produced by the interaction of a released moiety, such as a released ion interacting with an ion sensitive layer, or a pyrophosphate reacting with an enzyme or other catalyst to produce a chemiluminescent signal. In another example, changes in an electrical current, voltage, or resistance are detected without the need for an illumination source. Various illumination sources are discussed in detail below.

In some embodiments, a data acquisition analysis and control unit monitors various system parameters. The system parameters can include temperature of various portions of the instrument, such as sample processing unit or reagent reservoirs, volumes of various reagents, the status of various system subcomponents, such as a manipulator, a stepper motor, a pump, or the like, or any combination thereof.

It will be appreciated by one skilled in the art that the various embodiments of the instruments and systems used to practice sequencing methods such as sequencing by synthesis, single molecule methods, and other sequencing techniques, can be used with the CHAMP methods and platform described herein.

The methods and arrays disclosed herein for use with CHAMP methods and platforms can include high throughput sequencing chips, and preferably next generation sequencing technologies, as understood by those of skill in the art, which are useful with the CHAMP method and platform, as disclosed herein. Suitable high throughput sequencing methods and apparatus that fall within the scope of the invention include, but are not restricted to Solexa® or Illumina® sequencing by the detection of fluorescent dye labelled nucleotides with reversible terminator, and Pacific Bioscience Single molecule real time sequencing (SMRT). Other non-polymerase based DNA sequencing methods include SOLiD sequencing (Sequencing by Oligonucleotide Ligation and Detection), and sequencing by hybridization (SBH). These are described in more detail below.

In the Solexa/Illumina® platform (Voelkerding et al., Clinical Chem., 55: 641-658, 2009; MacLean et al., Nature Rev. Microbiol., 7: 287-296; U.S. Pat. Nos. 6,833,246; 7,115,400; 6,969,488; each herein incorporated by reference in its entirety), sequencing data are produced in the form of shorter-length reads. In this method, the fragments of the NGS fragment library are captured on the surface of a flow cell that is studded with oligonucleotide anchors. The anchor is used as a PCR primer, but because of the length of the template and its proximity to other nearby anchor oligonucleotides, extension by PCR results in the “arching over” of the molecule to hybridize with an adjacent anchor oligonucleotide to form a bridge structure on the surface of the flow cell. These loops of DNA are denatured and cleaved. Forward strands are then sequenced with reversible dye terminators. The sequence of incorporated nucleotides is determined by detection of post-incorporation fluorescence, with each fluor and block removed prior to the next cycle of dNTP addition. Sequence read length ranges from 36 nucleotides to over 100 nucleotides, with overall output exceeding 1 billion nucleotide pairs per analytical run.

Sequencing nucleic acid molecules using SOLiD technology (Voelkerding et al., Clinical Chem., 55: 641-658, 2009; MacLean et al., Nature Rev. Microbiol., 7: 287-296; U.S. Pat. Nos. 5,912,148; 6,130,073; each herein incorporated by reference in their entirety) also involves clonal amplification of the NGS fragment library by emulsion PCR. Following this, beads bearing template are immobilized on a derivatized surface of a glass flow-cell, and a primer complementary to the adaptor oligonucleotide is annealed. However, rather than utilizing this primer for 3′ extension, it is instead used to provide a 5′ phosphate group for ligation to interrogation probes containing two probe-specific bases followed by 6 degenerate bases and one of four fluorescent labels. In the SOLiD system, interrogation probes have 16 possible combinations of the two bases at the 3′ end of each probe, and one of four fluors at the 5′ end. Fluor color, and thus identity of each probe, corresponds to specified color-space coding schemes. Multiple rounds (usually 7) of probe annealing, ligation, and fluor detection are followed by denaturation, and then a second round of sequencing using a primer that is offset by one base relative to the initial primer. In this manner, the template sequence can be computationally re-constructed, and template bases are interrogated twice, resulting in increased accuracy. Sequence read length averages 35 nucleotides, and overall output exceeds 4 billion bases per sequencing run.

In certain embodiments, HeliScope® by Helicos BioSciences is employed (Voelkerding et al., Clinical Chem., 55: 641-658, 2009; MacLean et al., Nature Rev. Microbiol., 7: 287-296; U.S. Pat. Nos. 7,169,560; 7,282,337; 7,482,120; 7,501,245; 6,818,395; 6,911,345; 7,501,245; each herein incorporated by reference in their entirety). Sequencing is achieved by addition of polymerase and serial addition of fluorescently-labeled dNTP reagents. Incorporation events result in a fluor signal corresponding to the dNTP, and signal is captured by a CCD camera before each round of dNTP addition. Sequence read length ranges from 25-50 nucleotides, with overall output exceeding 1 billion nucleotide pairs per analytical run.

In some embodiments, 454 sequencing by Roche is used (Margulies et al. (2005) Nature 437: 376-380). 454 sequencing involves two steps. In the first step, DNA is sheared into fragments of approximately 300-800 base pairs and the fragments are blunt ended. Oligonucleotide adaptors are then ligated to the ends of the fragments. The adaptors serve as primers for amplification and sequencing of the fragments. The fragments can be attached to DNA capture beads, e.g., streptavidin-coated beads using, e.g., an adaptor that contains a 5′-biotin tag. The fragments attached to the beads are PCR amplified within droplets of an oil-water emulsion. The result is multiple copies of clonally amplified DNA fragments on each bead. In the second step, the beads are captured in wells (pico-liter sized). Pyrosequencing is performed on each DNA fragment in parallel. Addition of one or more nucleotides generates a light signal that is recorded by a CCD camera in a sequencing instrument. The signal strength is proportional to the number of nucleotides incorporated. Pyrosequencing makes use of pyrophosphate (PPi) which is released upon nucleotide addition. PPi is converted to ATP by ATP sulfurylase in the presence of adenosine 5′ phosphosulfate. Luciferase uses ATP to convert luciferin to oxyluciferin, and this reaction generates light that is detected and analyzed.

The Ion Torrent technology is a method of DNA sequencing based on the detection of hydrogen ions that are released during the polymerization of DNA (see. e.g., Science 327(5970): 1190 (2010); U.S. Pat. Appl. Pub. Nos. 20090026082, 20090127589, 20100301398, 20100197507, 20100188073, and 20100137143, incorporated by reference in their entireties for all purposes). A microwell contains a fragment of the NGS fragment library to be sequenced. Beneath the layer of microwells is a hypersensitive ISFET ion sensor. All layers are contained within a CMOS semiconductor chip, similar to that used in the electronics industry. When a dNTP is incorporated into the growing complementary strand a hydrogen ion is released, which triggers a hypersensitive ion sensor. If homopolymer repeats are present in the template sequence, multiple dNTP molecules will be incorporated in a single cycle. This leads to a corresponding number of released hydrogens and a proportionally higher electronic signal. This technology differs from other sequencing technologies in that no modified nucleotides or optics are used. The per-base accuracy of the Ion Torrent sequencer is 99.6% for 50 base reads, with 100 Mb generated per run. The read-length is 100 base pairs. The accuracy for homopolymer repeats of 5 repeats in length is 98%.

Another exemplary nucleic acid sequencing approach that may be adapted for use with the present invention was developed by Stratos Genomics, Inc, and involves the use of Xpandomers. This sequencing process typically includes providing a daughter strand produced by a template-directed synthesis. The daughter strand generally includes a plurality of subunits coupled in a sequence corresponding to a contiguous nucleotide sequence of all or a portion of a target nucleic acid in which the individual subunits comprise a tether, at least one probe or nucleobase residue, and at least one selectively cleavable bond. The selectively cleavable bond(s) is/are cleaved to yield an Xpandomer of a length longer than the plurality of the subunits of the daughter strand. The Xpandomer typically includes the tethers and reporter elements for parsing genetic information in a sequence corresponding to the contiguous nucleotide sequence of all or a portion of the target nucleic acid. Reporter elements of the Xpandomer are then detected. Additional details relating to Xpandomer-based approaches are described in, for example, U.S. Pat. Pub No. 20090035777, entitled “HIGH THROUGHPUT NUCLEIC ACID SEQUENCING BY EXPANSION,” filed Jun. 19, 2008, which is incorporated herein in its entirety.

Other single molecule sequencing methods useful with the CHAMP platform include real-time sequencing by synthesis using a VisiGen platform (Voelkerding et al., Clinical Chem., 55: 641-58, 2009; U.S. Pat. No. 7,329,492; U.S. patent application Ser. No. 11/671,956; U.S. patent application Ser. No. 11/781,166; each herein incorporated by reference in their entirety) in which fragments of the NGS fragment library are immobilized, primed, then subjected to strand extension using a fluorescently-modified polymerase and florescent acceptor molecules, resulting in detectable fluorescence resonance energy transfer (FRET) upon nucleotide addition.

Another real-time single molecule sequencing system developed by Pacific Biosciences (Voelkerding et al., Clinical Chem., 55: 641-658, 2009; MacLean et al., Nature Rev. Microbiol., 7: 287-296; U.S. Pat. Nos. 7,170,050; 7,302,146; 7,313,308; 7,476,503; all of which are herein incorporated by reference) utilizes reaction wells 50-100 nm in diameter and encompassing a reaction volume of approximately 20 zeptoliters (10-21 l). Sequencing reactions are performed using immobilized template, modified phi29 DNA polymerase, and high local concentrations of fluorescently labeled dNTPs. High local concentrations and continuous reaction conditions allow incorporation events to be captured in real time by fluor signal detection using laser excitation, an optical waveguide, and a CCD camera.

In certain embodiments, the single molecule real time (SMRT) DNA sequencing methods using zero-mode waveguides (ZMWs) developed by Pacific Biosciences, or similar methods, are employed. With this technology, DNA sequencing is performed on SMRT chips, each containing thousands of zero-mode waveguides (ZMWs). A ZMW is a hole, tens of nanometers in diameter, fabricated in a 100 nm metal film deposited on a silicon dioxide substrate. Each ZMW becomes a nanophotonic visualization chamber providing a detection volume of just 20 zeptoliters (10-21 l). At this volume, the activity of a single molecule can be detected amongst a background of thousands of labeled nucleotides. The ZMW provides a window for watching DNA polymerase as it performs sequencing by synthesis. Within each chamber, a single DNA polymerase molecule is attached to the bottom surface such that it permanently resides within the detection volume. Phospholinked nucleotides, each type labeled with a different colored fluorophore, are then introduced into the reaction solution at high concentrations which promote enzyme speed, accuracy, and processivity. Due to the small size of the ZMW, even at these high, biologically relevant concentrations, the detection volume is occupied by nucleotides only a small fraction of the time. In addition, visits to the detection volume are fast, lasting only a few microseconds, due to the very small distance that diffusion has to carry the nucleotides. The result is a very low background.

In some embodiments, nanopore sequencing can be used with the disclosed methods and platforms (Soni G V and Meller A. (2007) Clin Chem 53: 1996-2001). A nanopore is a small hole, of the order of 1 nanometer in diameter. Immersion of a nanopore in a conducting fluid and application of a potential across it results in a slight electrical current due to conduction of ions through the nanopore. The amount of current which flows is sensitive to the size of the nanopore. As a DNA molecule passes through a nanopore, each nucleotide on the DNA molecule obstructs the nanopore to a different degree. Thus, the change in the current passing through the nanopore as the DNA molecule passes through the nanopore represents a reading of the DNA sequence.

In some embodiments, a sequencing technique uses a chemical-sensitive field effect transistor (chemFET) array to sequence DNA (for example, as described in US Patent Application Publication No. 20090026082). In one example of the technique, DNA molecules are placed into reaction chambers, and the template molecules are hybridized to a sequencing primer bound to a polymerase. Incorporation of one or more triphosphates into a new nucleic acid strand at the 3′ end of the sequencing primer can be detected by a change in current by a chemFET. An array can have multiple chemFET sensors. In another example, single nucleic acids can be attached to beads, and the nucleic acids can be amplified on the bead, and the individual beads can be transferred to individual reaction chambers on a chemFET array, with each chamber having a chemFET sensor, and the nucleic acids can be sequenced.

In some embodiments, “four-color sequencing by synthesis using cleavable fluorescents nucleotide reversible terminators” as described in Turro, et al. PNAS 103: 19635-40 (2006) is used, e.g., as commercialized by Intelligent Bio-Systems for sequencing prior to CHAMP. The technology described in U.S. Pat. Appl. Pub. Nos. 2010/0323350, 2010/0063743, 2010/0159531, 20100035253, 20100152050, incorporated herein by reference for all purposes.

Processes and systems for such real time sequencing that may be adapted for use with the invention are described in, for example, U.S. Pat. No. 7,405,281, entitled “Fluorescent nucleotide analogs and uses therefor”, issued Jul. 29, 2008 to Xu et al.; U.S. Pat. No. 7,315,019, entitled “Arrays of optical confinements and uses thereof”, issued Jan. 1, 2008 to Turner et al.; U.S. Pat. No. 7,313,308, entitled “Optical analysis of molecules”, issued Dec. 25, 2007 to Turner et al.; U.S. Pat. No. 7,302,146, entitled “Apparatus and method for analysis of molecules”, issued Nov. 27, 2007 to Turner et al.; and U.S. Pat. No. 7,170,050, entitled “Apparatus and methods for optical analysis of molecules”, issued Jan. 30, 2007 to Turner et al.; and U.S. Pat. Pub. Nos. 20080212960, entitled “Methods and systems for simultaneous real-time monitoring of optical signals from multiple sources”, filed Oct. 26, 2007 by Lundquist et al.; 20080206764, entitled “Flowcell system for single molecule detection”, filed Oct. 26, 2007 by Williams et al.; 20080199932, entitled “Active surface coupled polymerases”, filed Oct. 26, 2007 by Hanzel et al.; 20080199874, entitled “CONTROLLABLE STRAND SCISSION OF MINI CIRCLE DNA”, filed Feb. 11, 2008 by Otto et al.; 20080176769, entitled “Articles having localized molecules disposed thereon and methods of producing same”, filed Oct. 26, 2007 by Rank et al.; 20080176316, entitled “Mitigation of photodamage in analytical reactions”, filed Oct. 31, 2007 by Eid et al.; 20080176241, entitled “Mitigation of photodamage in analytical reactions”, filed Oct. 31, 2007 by Eid et al.; 20080165346, entitled “Methods and systems for simultaneous real-time monitoring of optical signals from multiple sources”, filed Oct. 26, 2007 by Lundquist et al.; 20080160531, entitled “Uniform surfaces for hybrid material substrates and methods for making and using same”, filed Oct. 31, 2007 by Korlach; 20080157005, entitled “Methods and systems for simultaneous real-time monitoring of optical signals from multiple sources”, filed Oct. 26, 2007 by Lundquist et al.; 20080153100, entitled “Articles having localized molecules disposed thereon and methods of producing same”, filed Oct. 31, 2007 by Rank et al.; 20080153095, entitled “CHARGE SWITCH NUCLEOTIDES”, filed Oct. 26, 2007 by Williams et al.; 20080152281, entitled “Substrates, systems and methods for analyzing materials”, filed Oct. 31, 2007 by Lundquist et al.; 20080152280, entitled “Substrates, systems and methods for analyzing materials”, filed Oct. 31, 2007 by Lundquist et al.; 20080145278, entitled “Uniform surfaces for hybrid material substrates and methods for making and using same”, filed Oct. 31, 2007 by Korlach; 20080128627, entitled “SUBSTRATES. SYSTEMS AND METHODS FOR ANALYZING MATERIALS”, filed Aug. 31, 2007 by Lundquist et al.; 20080108082, entitled “Polymerase enzymes and reagents for enhanced nucleic acid sequencing”, filed Oct. 22, 2007 by Rank et al.; 20080095488, entitled “SUBSTRATES FOR PERFORMING ANALYTICAL REACTIONS”, filed Jun. 11, 2007 by Foquet et al.; 20080080059, entitled “MODULAR OPTICAL COMPONENTS AND SYSTEMS INCORPORATING SAME”, filed Sep. 27, 2007 by Dixon et al.; 20080050747, entitled “Articles having localized molecules disposed thereon and methods of producing and using same”, filed Aug. 14, 2007 by Korlach et al.; 20080032301, entitled “Articles having localized molecules disposed thereon and methods of producing same”, filed Mar. 29, 2007 by Rank et al.; 20080030628, entitled “Methods and systems for simultaneous real-time monitoring of optical signals from multiple sources”, filed Feb. 9, 2007 by Lundquist et al.; 20080009007, entitled “CONTROLLED INITIATION OF PRIMER EXTENSION”, filed Jun. 15, 2007 by Lyle et al.; 20070238679, entitled “Articles having localized molecules disposed thereon and methods of producing same”, filed Mar. 30, 2006 by Rank et al.; 20070231804, entitled “Methods, systems and compositions for monitoring enzyme activity and applications thereof”, filed Mar. 31, 2006 by Korlach et al.; 20070206187, entitled “Methods and systems for simultaneous real-time monitoring of optical signals from multiple sources”, filed Feb. 9, 2007 by Lundquist et al.; 20070196846, entitled “Polymerases for nucleotide analog incorporation”, filed Dec. 21, 2006 by Hanzel et al.; 20070188750, entitled “Methods and systems for simultaneous real-time monitoring of optical signals from multiple sources”, filed Jul. 7, 2006 by Lundquist et al.; 20070161017, entitled “MITIGATION OF PHOTODAMAGE IN ANALYTICAL REACTIONS”, filed Dec. 1, 2006 by Eid et al.; 20070141598, entitled “Nucleotide Compositions and Uses Thereof”, filed Nov. 3, 2006 by Turner et al.; 20070134128, entitled “Uniform surfaces for hybrid material substrate and methods for making and using same”, filed Nov. 27, 2006 by Korlach; 20070128133, entitled “Mitigation of photodamage in analytical reactions”, filed Dec. 2, 2005 by Eid et al.; 20070077564, entitled “Reactive surfaces, substrates and methods of producing same”, filed Sep. 30, 2005 by Roitman et al.; 20070072196, entitled “Fluorescent nucleotide analogs and uses therefore”, filed Sep. 29, 2005 by Xu et al; and 20070036511, entitled “Methods and systems for monitoring multiple optical signals from a single source”, filed Aug. 11, 2005 by Lundquist et al.; and Korlach et al. (2008) “Selective aluminum passivation for targeted immobilization of single DNA polymerase molecules in zero-mode waveguide nanostructures” PNAS 105(4): 1176-81, all of which are herein incorporated by reference in their entireties.

b) Proteins

Proteins/peptide sequences capable of being used with the methods and assays described herein are not limited. For example, proteins can be used which bind nonspecifically to a nucleic acid or to a specific nucleic acid sequence, such as proteins which regulate gene expression and/or activity. The protein can either be a functional protein or a protein fragment. Proteins can also be simple proteins, which are composed of only amino acids, and conjugated proteins, which are composed of amino acids and additional organic and inorganic groupings, certain of which are called prosthetic groups. Conjugated proteins include glycoproteins, which contain carbohydrates; lipoproteins, which contain lipids; and nucleoproteins, which contain nucleic acids. As above, the identity of the protein need not be known when interacted with the nucleic acid and can be determined at a later point through known techniques, In fact, the present invention can be used to identify novel proteins and characterize their interactions with nucleic acid. Different proteins can also be used in different iterations of the present method using the same nucleic acid. Related proteins can also be used in these iterations to determine the effect mutations in the protein have on the measured interactions. Likewise, proteins having a known mutation can be tested in parallel with the wild-type protein to determine the possible effects the protein mutation has on nucleic acid-protein interactions.
c) Labeling/Detection of Nucleic Acid-Protein Interaction

Preferably, either the nucleic acid, protein or both are labeled. Suitable labels include ligands which bind to labeled antibodies, fluorophores, chemiluminescent agents, enzymes, and antibodies which can serve as specific binding pair members for a labeled ligand. Fluorescence quenching labeling schemes can also be used in the present methods, wherein one of the protein or nucleic acid is labeled with a fluorescent moiety and the other is labeled with a quenching moiety such that interaction of the two results in fluorescent quenching. One or more labels can also be incorporated onto the nucleic acid and/or protein. This can be useful when a nucleic acid of significant length used in order to determine where the protein interacts with the nucleic acid. Multiple labels on the protein can also provide an indication about which part of the protein interacts with the nucleic acid.

The label may also allow for the indirect detection of the hybridization complex. For example, where the label is a hapten or antigen, the sample can be detected by using antibodies. In these systems, a signal is generated by attaching fluorescent or enzyme molecules to the antibodies or, in some cases, by attachment to a radioactive label. (Tijssen, “Practice and Theory of Enzyme Immunoassays,” Laboratory Techniques in Biochemistry and Molecular Biology” (Burdon, van Knippenberg (eds.). Elsevier, pp. 9-20 (1985)).

Useful labels in the present invention include biotin for staining with labeled streptavidin conjugate, fluorescent dyes (e.g., fluorescein, texas red, rhodamine, green fluorescent protein, and the like), radiolabels (e.g., ³H, ¹²⁵I, ³⁵S, ¹⁴C, and ³²P), and enzymes (e.g., horse radish peroxidase, alkaline phosphatase and others commonly used in an ELISA). Patents teaching the use of such labels include U.S. Pat. Nos. 3,817,837; 3,850,752; 3,939,350; 3,996,345; 4,277,437; 4,275,149; and 4,366,241.

Means of detecting such labels are well known to those of skill in the art. Thus, for example, radiolabels may be detected using photographic film or scintillation counters, fluorescent markers may be detected using a photodetector to detect emitted light. Enzymatic labels are typically detected by providing the enzyme with a substrate and detecting the reaction product produced by the action of the enzyme on the substrate, and calorimetric labels are detected by simply visualizing the colored label.

The interaction between the nucleic acid and protein can be characterized by any means known in the art. Preferably, the interaction is characterized by measuring an event which causes or quenches fluorescence. Alternatively, the strength of the interaction can be determined by measuring the melting temperature of the nucleic acid or the temperature which causes dissociation of the protein from the nucleic acid.

The subject methods of identifying protein/nucleic acid binding pairs can be used in a variety of different applications. Representative applications of interest include research applications, where the subject invention is employed to identify and characterize protein/nucleic acid binding pairs. As such, one can employ the subject invention to rapidly identify and characterize RNA/protein binding pairs, single-stranded DNA/protein binding pairs (where the protein members may be involved in DNA replication, repair, recombination, etc.), double-stranded DNA/protein binding pairs (where the protein members may be histones, transcription factors, methylases, polymerases, etc.), telomeric DNA/protein binding pairs, secondary structure (e.g., Z-DNA. G-quartet DNA, triplex DNA, cruciforms, etc.) assuming nucleic acid/protein binding pairs, etc., in various research applications, such as elucidation of biochemical pathways, e.g., cellular processes such as replication, transcription, signaling, etc.

A variety of illumination systems may be used with the present methods and arrays. The illumination systems can comprise lamps and/or lasers. In particular embodiments, excitation generated from a lamp or laser can be optically filtered to select a desired wavelength for illumination of a sample. The systems can contain one or more illumination lasers of different wavelengths. In one example, illumination of fluorescence is performed using Total Internal Reflection (TIR) comprising a laser component. It will be appreciated that a “TIRF laser,” “TIRF laser system,” “TIR laser,” and other similar terminology herein refers to a TIRF (Total Internal Reflection Fluorescence) based detection instrument/system using excitation, e.g., lasers or other types of non-laser excitation from such light sources as LED, halogen, and xenon or mercury arc lamps (all of which are also included in the current description of TIRF, TIRF laser, TIRF laser system, etc, herein). Thus, a “TIRF laser” is a laser used with a TIRF system, while a “TIRF laser system” is a TIRF system using a laser, etc. Again, however, the systems herein (even when described in terms of having laser usage, etc.) should also be understood to include those systems/instruments comprising non-laser based excitation sources. In some embodiments, the laser comprises dual individually modulated 50 mW to 500 mW solid state and/or semiconductor lasers coupled to a TIRF prism, optionally with excitation wavelengths of 532 nm and 660 nm. The coupling of the laser into the instrument can be via an optical fiber to help ensure that the footprints of the two lasers are focused on the same or common area of the substrate (i.e., overlap).

Multi-color co-localization can used to determine protein-nucleic acid interaction. An example of using multi-color colocalization can be found in U.S. Pat. No. 6,844,150, herein incorporated by reference in its entirety. Time-dependent kinetics of protein-nucleic acid interactions can also be measured using the methods disclosed herein. An example of time-dependent kinetics can be found in U.S. Pat. No. 6,589,729, herein incorporated by reference in its entirety. Protein or nucleic acid conformations can be measured via Förster resonance energy transfer (FRET) or other fluorescence transfer or quenching methods. An example of FRET can be found in U.S. Pat. No. 6,908,769 herein incorporated by reference in its entirety

d) Systems

Disclosed herein is a system for use with the CHAMP method and platform. The system can include a nucleic acid-protein interaction identification means, data storage, reference sequence data storage, and an analytics computing device/server/node. In some embodiments, the analytics computing device/server/node can be a workstation, mainframe computer, personal computer, mobile device, etc. The nucleic acid-protein interaction identification means can be configured to analyze (e.g., interrogate) a nucleic acid and protein interaction. This can be done utilizing all available varieties of techniques, platforms or technologies to obtain sequence information and protein interaction information, in particular the methods as described herein using compositions provided herein. In some embodiments, the nucleic acid-protein interaction identification means is in communication with sequence data storage obtained during the sequencing phase, either directly via a data cable (e.g., serial cable, direct cable connection, etc.) or bus linkage or, alternatively, through a network connection (e.g., Internet, LAN, WAN, VPN, etc.). In some embodiments, the network connection can be a “hardwired” physical connection.

In some embodiments, the sequence data storage is any database storage device, system, or implementation (e.g., data storage partition, etc.) that is configured to organize and store nucleic acid sequence read data generated by nucleic acid sequencer such that the data can be searched and retrieved manually (e.g., by a database administrator or client operator) or automatically by way of a computer program, application, or software script. In some embodiments, the reference data storage can be any database device, storage system, or implementation (e.g., data storage partition, etc.) that is configured to organize and store reference sequences (e.g., whole or partial genome, whole or partial exome, SNP, gen, etc.) such that the data can be searched and retrieved manually (e.g., by a database administrator or client operator) or automatically by way of a computer program, application, and/or software script. In some embodiments, the sample nucleic acid sequencing read data can be stored on the sample sequence data storage and/or the reference data storage in a variety of different data file types/formats, including, but not limited to: *.txt, *.fasta, *.csfasta, *seq.txt, *qseq.txt, *.fastq, *.sff, *prb.txt, *.sms, *srs and/or *.qv.

In some embodiments, the sequence data storage and the nucleic acid-protein interaction data storage are independent standalone devices/systems or implemented on different devices. In some embodiments, the sequence data storage and the nucleic acid-protein interaction data storage are implemented on the same device/system. In some embodiments, the sequence data storage and/or the nucleic acid-protein interaction data storage can be implemented on the analytics computing device/server/node. The analytics computing device/server/node can be in communications with the sequence data storage and the nucleic acid-protein interaction data storage either directly via a data cable (e.g., serial cable, direct cable connection, etc.) or bus linkage or, alternatively, through a network connection (e.g., Internet, LAN, WAN, VPN, etc.). In some embodiments, analytics computing device/server/node can host a reference mapping engine, a de novo mapping module, and/or a tertiary analysis engine.

In some embodiments, the reference mapping engine can be configured to obtain nucleic acid-protein interaction reads from the sample data storage and map them against one or more reference sequences obtained from the sequence data storage to assemble the reads using all varieties of reference mapping/alignment techniques and methods. It should be understood that the various engines and modules hosted on the analytics computing device/server/node can be combined or collapsed into a single engine or module, depending on the requirements of the particular application or system architecture. Moreover, in some embodiments, the analytics computing device/server/node can host additional engines or modules as needed by the particular application or system architecture.

In some embodiments, the mapping and/or tertiary analysis engines are configured to process the data in color space. In some embodiments, the mapping and/or tertiary analysis engines are configured to process the data in base space. It should be understood, however, that the mapping and/or tertiary analysis engines disclosed herein can process or analyze data in any schema or format as long as the schema or format can convey the base identity and position of the nucleic acid sequence.

In some embodiments, the obtained data can be supplied to the analytics computing device/server/node in a variety of different input data file types/formats, including, but not limited to: *.txt, *.fasta, *.csfasta, *seq.txt, *qseq.txt, *.fastq, *.sff, *prb.txt, *.sms, *srs and/or *.qv.

Furthermore, a client terminal can be a thin client or thick client computing device. In some embodiments, client terminal can have a web browser that can be used to control the operation of the reference mapping engine, the de novo mapping module and/or the tertiary analysis engine. That is, the client terminal can access the reference mapping engine, the de novo mapping module and/or the tertiary analysis engine using a browser to control their function. For example, the client terminal can be used to configure the operating parameters (e.g., mismatch constraint, quality value thresholds, etc.) of the various engines, depending on the requirements of the particular application. Similarly, client terminal can also display the results of the analysis performed by the reference mapping engine, the de novo mapping module and/or the tertiary analysis engine.

The present technology also encompasses any method capable of receiving, processing, and transmitting the information to and from laboratories conducting the assays, information provides, medical personal, and subjects.

### C. Examples

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1

Herein is described a chip-hybridized association-mapping platform (CHAMP) for comprehensively profiling protein-nucleic acid interactions on sequenced next generation sequencing (NGS) chips. The most widely adopted NGS sequencers fluorescently image clusters of DNA molecules covalently affixed to the surface of a microfluidic chip. CHAMP leverages these chips—which would normally be discarded after sequencing—to quantitatively measure protein-DNA interactions. Importantly. CHAMP does not require any hardware or software modifications to older NGS sequencers. Instead, it uses modern and ubiquitous Illumina instruments to generate chips and sequencing data. Protein-DNA profiling experiments are then performed independently on a standard fluorescence microscope. In short, NGS sequencing provides information about the position and identities of millions of different DNA molecules, while the microscopy experiments quantitatively measure binding interactions of the proteins to a library of DNA molecules.

CHAMP was used to quantitatively profile interactions between the T. fusca Type I-E CRISPR-Cas (Cascade) effector complex and a diverse library of genomic and synthetic target DNA molecules. Type I systems comprise approximately 50% of bacterial CRISPRs, and have been used to control gene expression and cell fate. CHAMP profiling revealed that Cascade recognizes an extended, six nucleotide protospacer adjacent motif (PAM). Quantitative profiling of off-target DNA-binding sequences reveals a three-nucleotide periodicity in Cascade-DNA interactions, observed in synthesized libraries and human genomic DNA. Cas3 recruitment was sensitive to the identity of the PAM and PAM-proximal DNA-RNA mismatches, establishing a novel DNA-guided proofreading mechanism. These results were used to develop a predictive biophysical framework that accurately reproduced in vivo interference experiments. Using CHAMP, CRISPR-Cas binding was profiled in human genomic DNA, paving the way for rapid and quantitative determination of off-target binding sites in patient-specific genomes. More broadly, this study provides an experimental and computational framework for comprehensive analysis of protein-DNA interactions for diverse CRISPR systems and other DNA-binding proteins on both synthetic and genomic DNA libraries.

a) Results

(1) A Chip-Hybridized Association-Mapping Platform (CHAMP) for Profiling CRISPR-Cas DNA Interactions

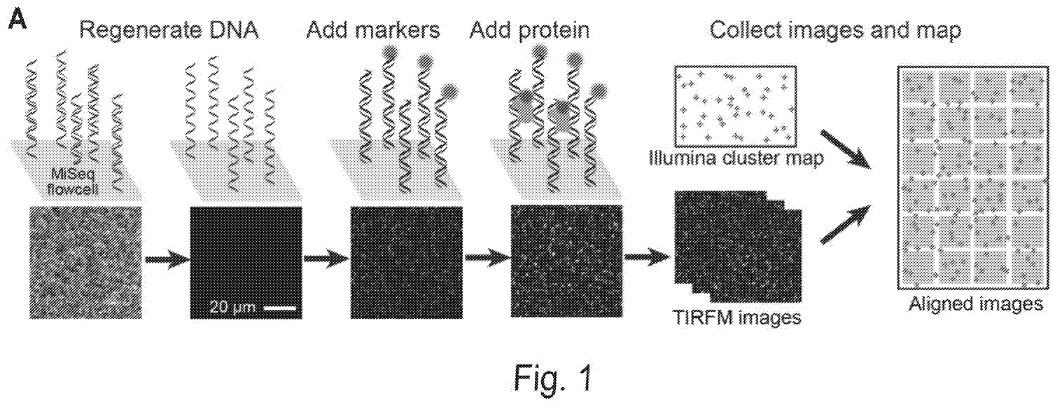

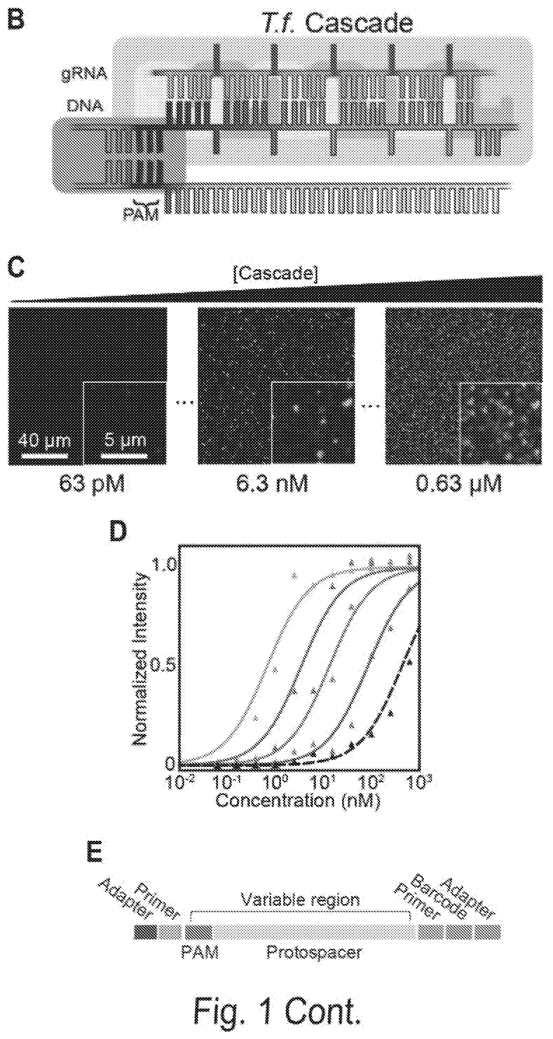

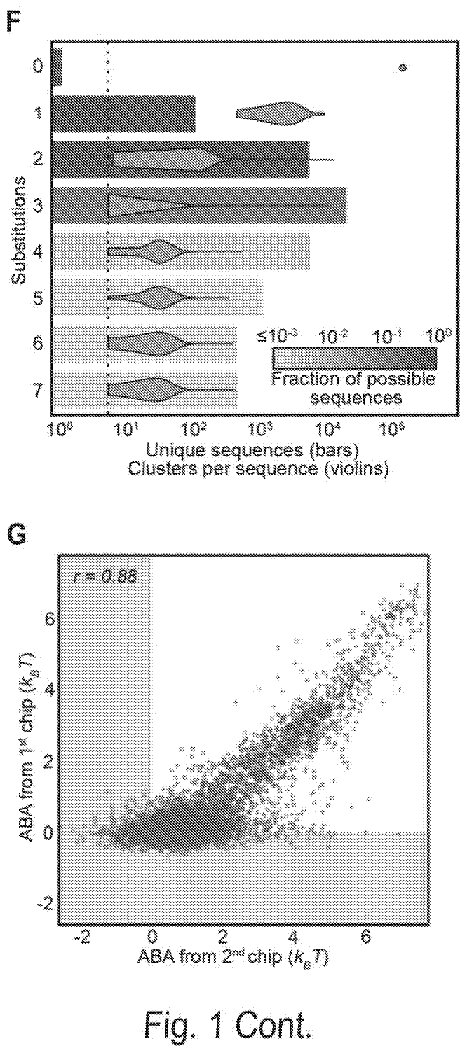

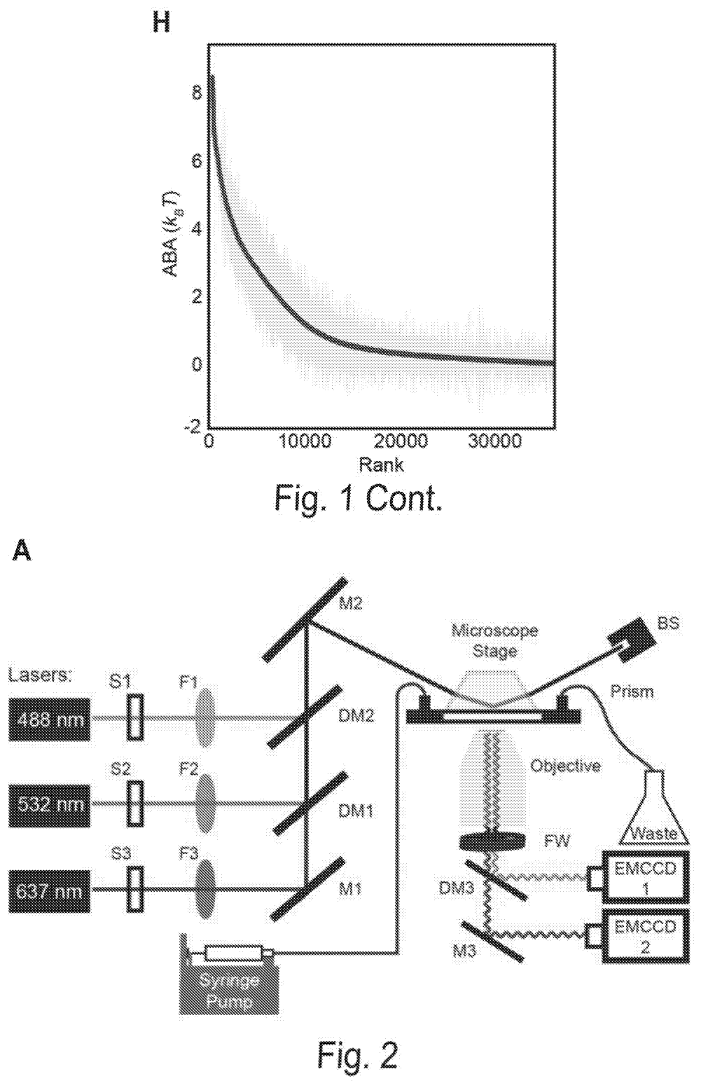

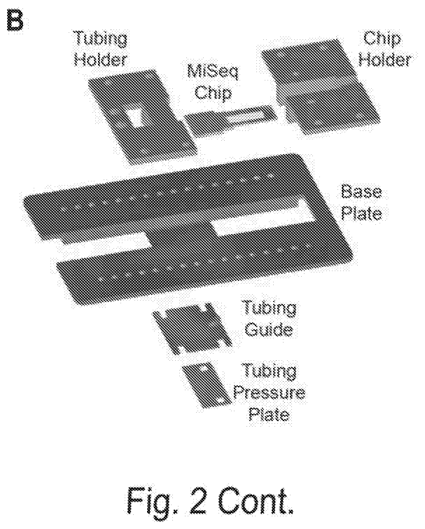

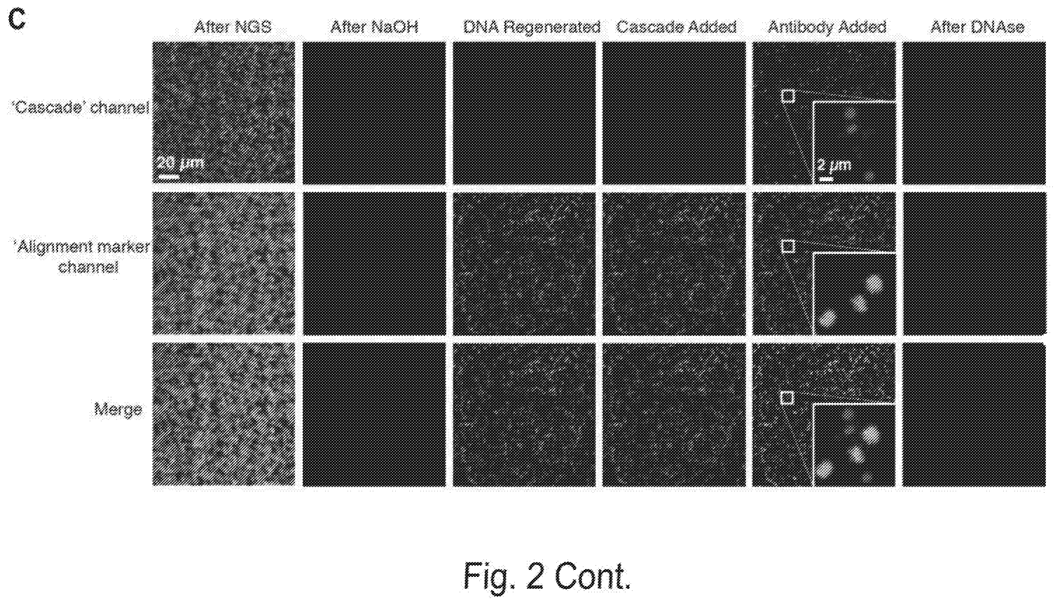

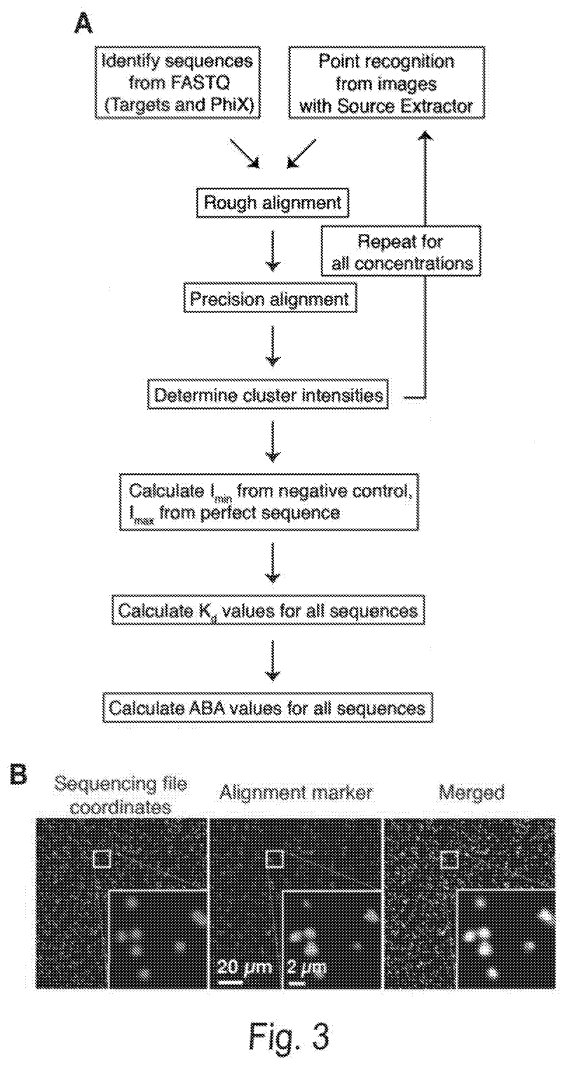

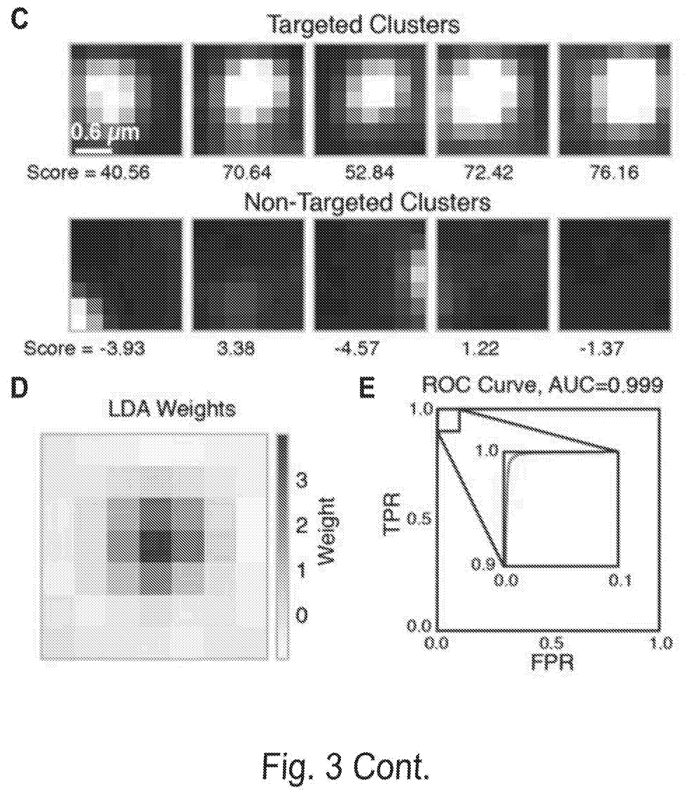

CHAMP leverages used MiSeq chips that are generated via the Illumina sequencing pipeline (FIG. 1). At the end of a DNA sequencing run, the surfaces of these chips are decorated with ˜20 million spatially registered, unique DNA clusters. CHAMP uses high-throughput fluorescence imaging to measure the association between fluorescently labeled protein complexes and each DNA cluster (FIG. 1A). The MiSeq sequencer is ubiquitous in nearly all NGS cores and genomics labs, produces long (˜300 bp) reads, and the MiSeq chips also contain integrated microfluidic ports. To prepare chips for CHAMP, the DNA clusters are first regenerated to remove any fluorescent nucleotides that can otherwise confound imaging (FIG. 2). A fluorescent oligonucleotide primer is then hybridized to a subset of the DNA clusters and used as an alignment marker in the downstream image-processing pipeline (FIG. 1A). Next, fluorescently labeled proteins are incubated in the chip and imaged using a total internal reflection fluorescence (TIRF) microscope. The images are then analyzed using the CHAMP software pipeline, which maps each fluorescent cluster to the underlying DNA sequence, as reported by the Illumina sequencer (FIG. 3 and Star Methods). CHAMP's strength lies in its platform independence and its software pipeline, which quantifies protein association with each DNA sequence (FIG. 1 and Star Methods).

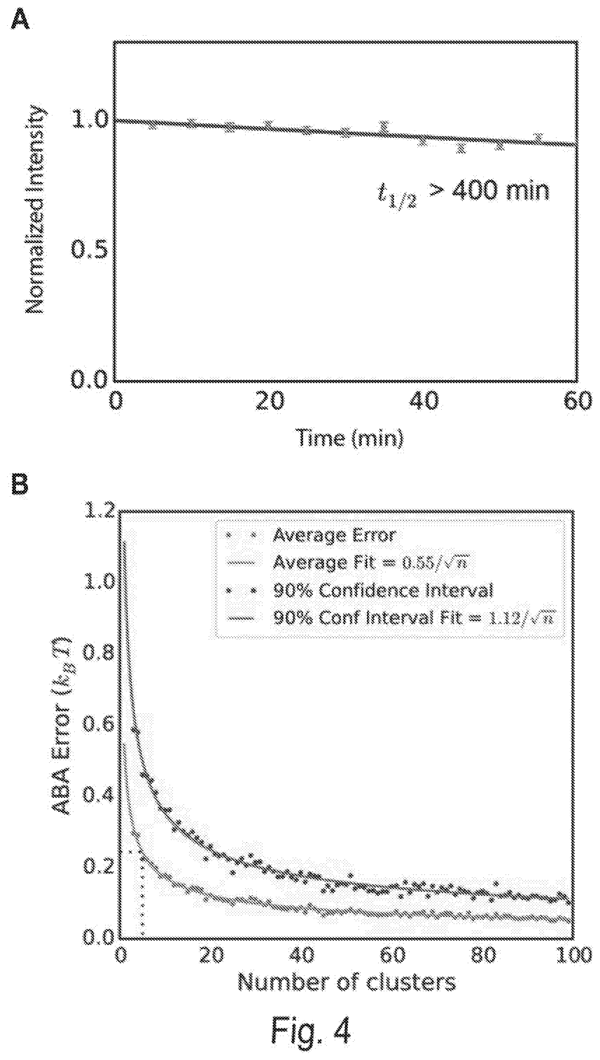

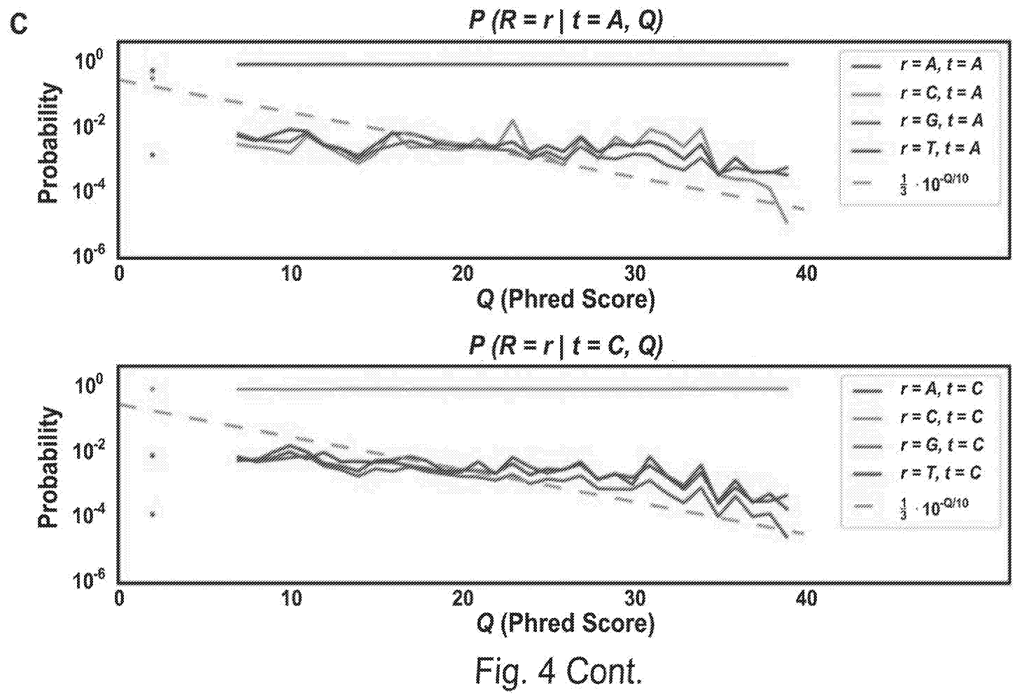

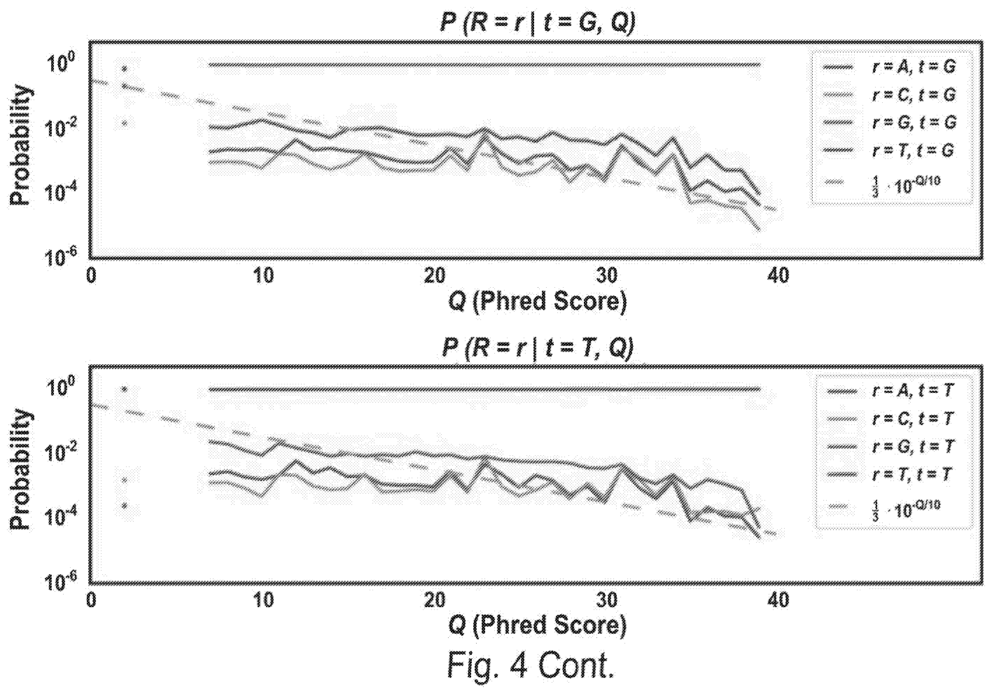

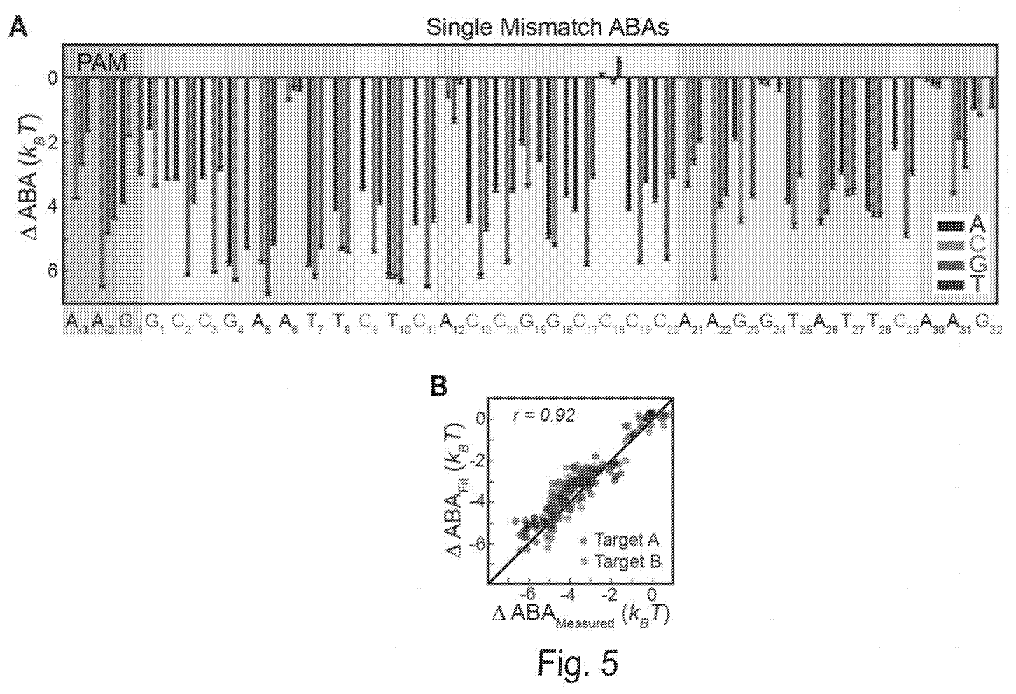

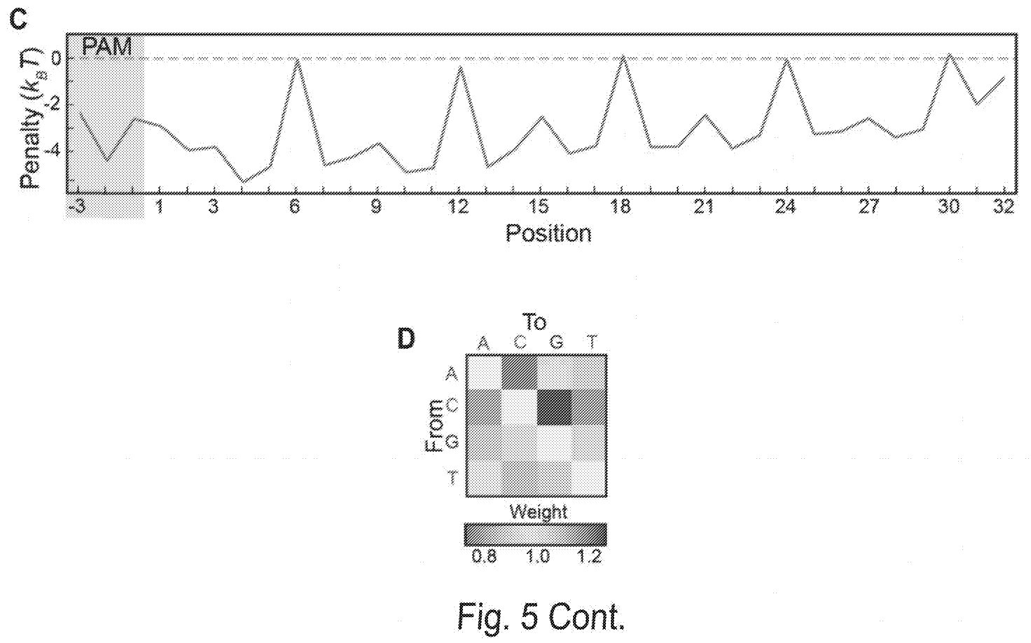

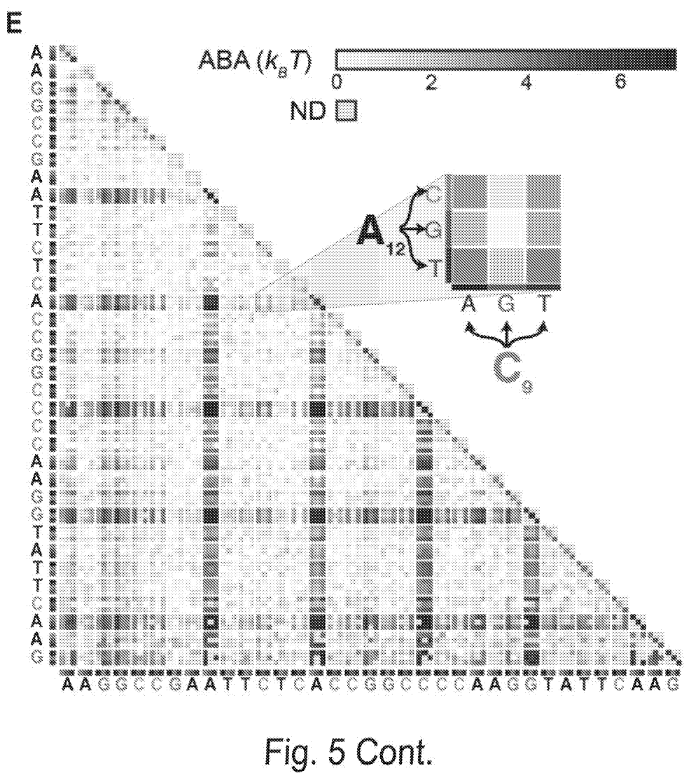

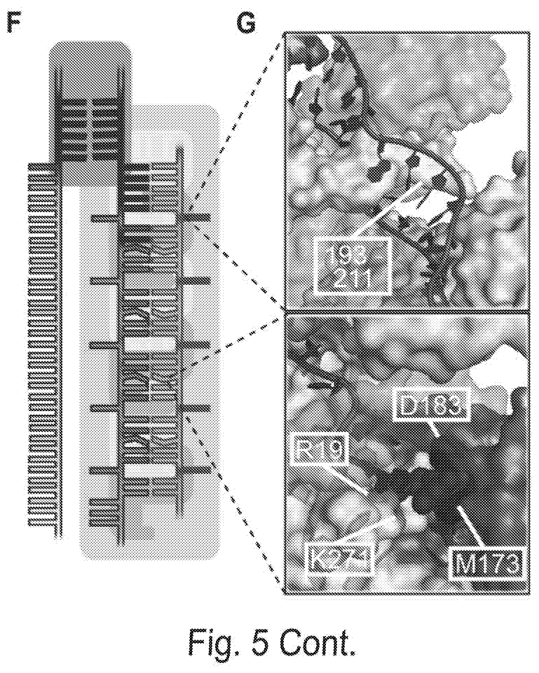

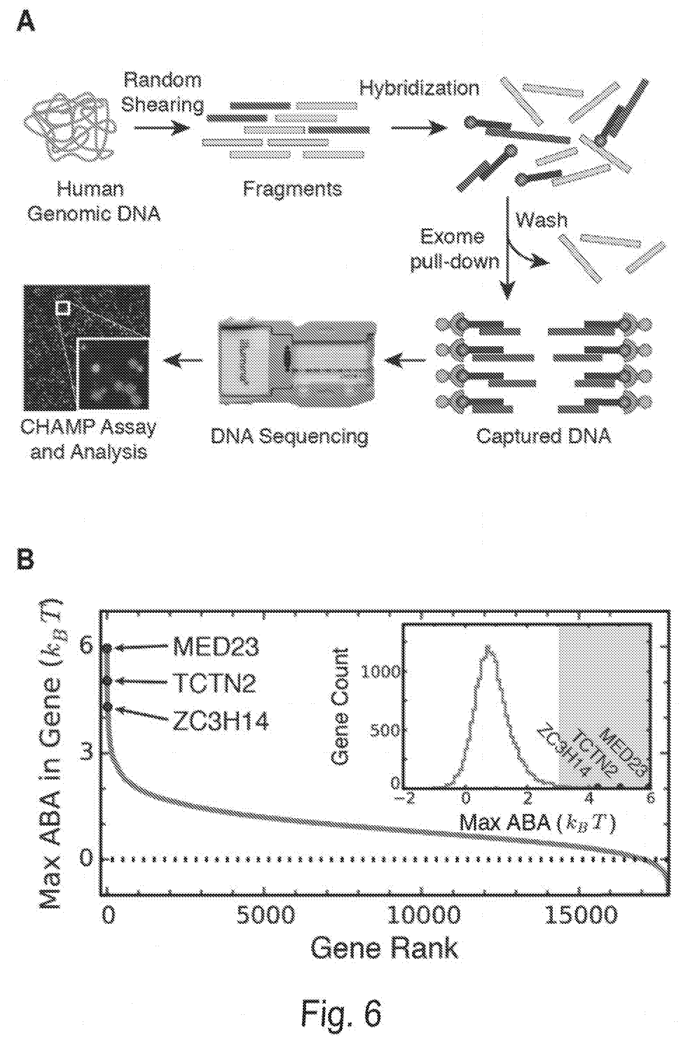

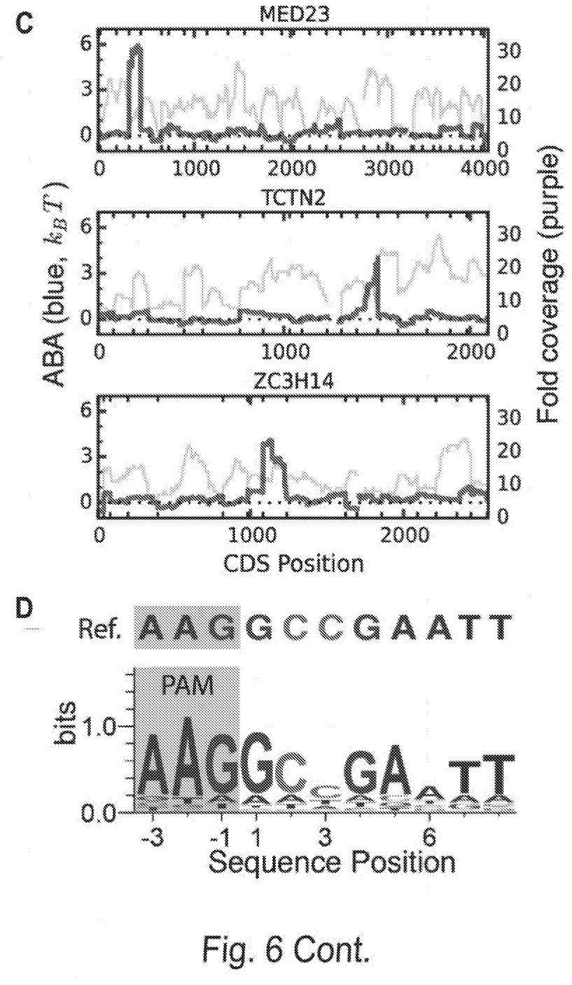

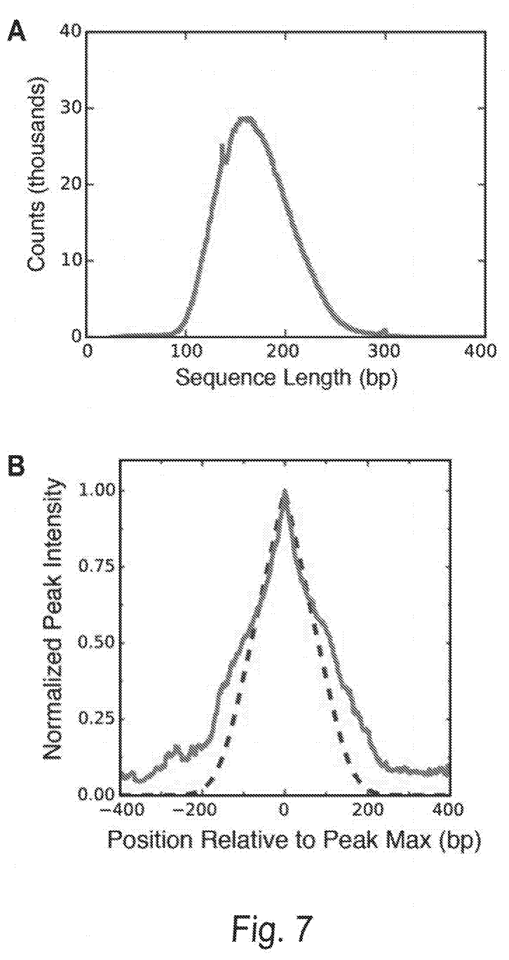

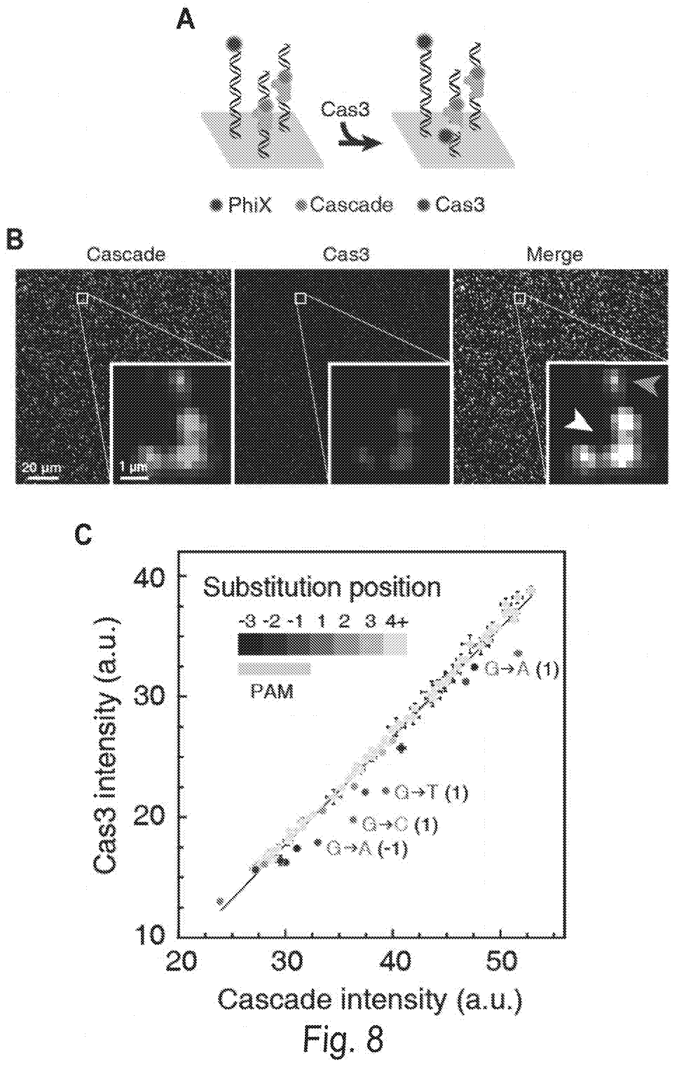

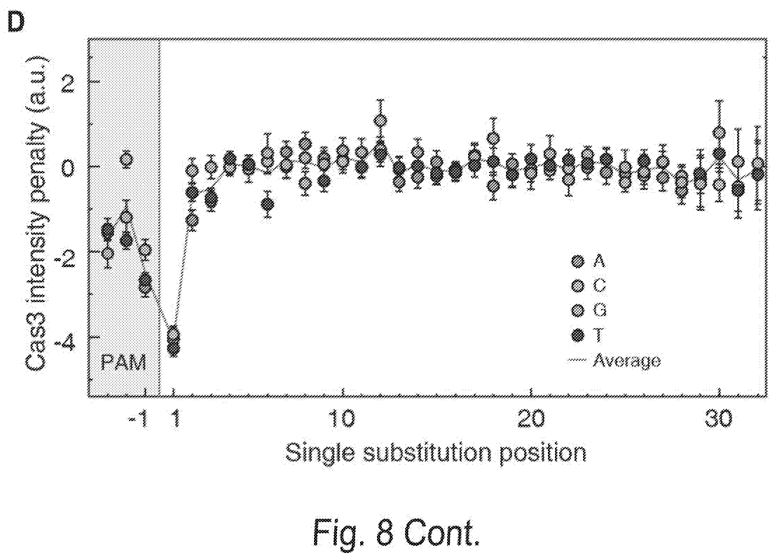

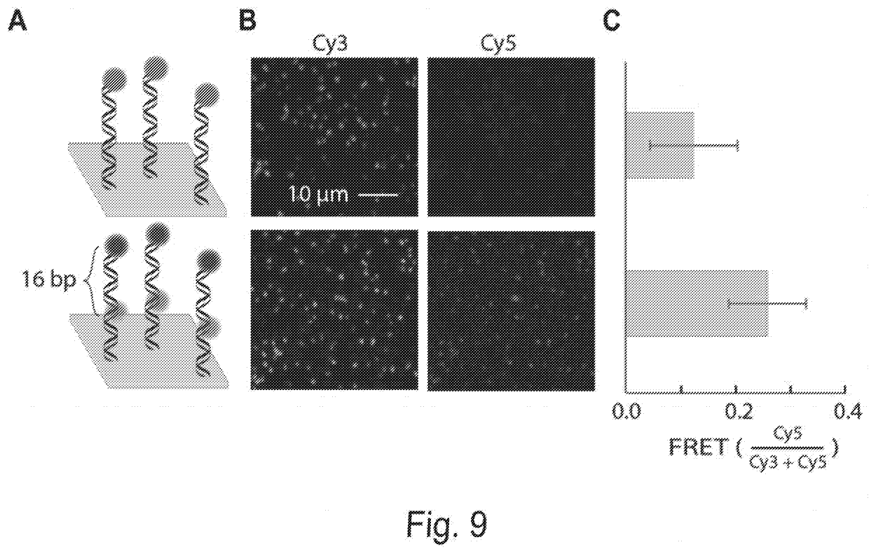

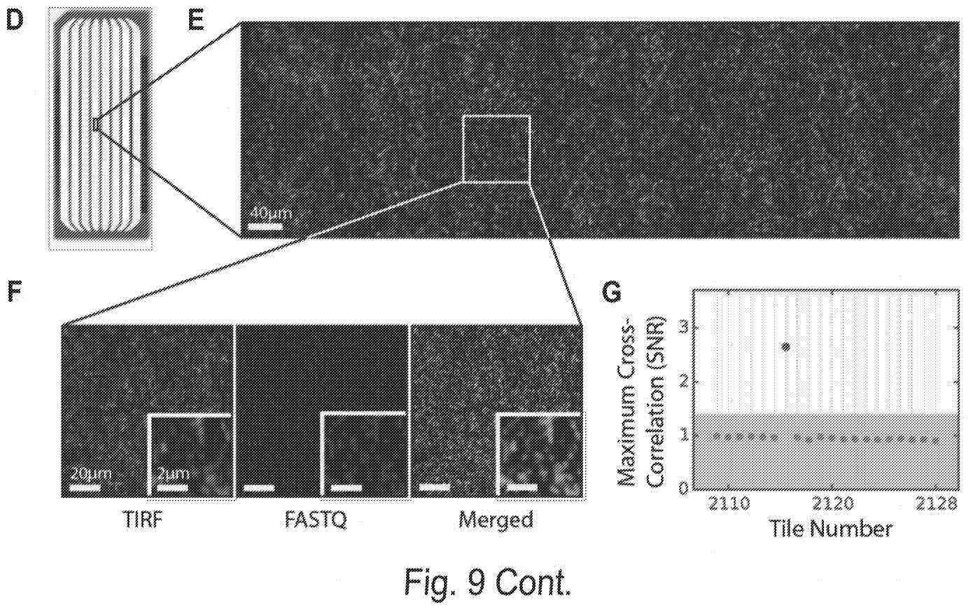

Using CHAMP, the PAM specificity and off-target binding affinity of the thermophilic T. fusca Type I-E CRISPR-Cas (Cascade) complex (FIG. 1B) was profiled. Experiments were carried out on regenerated MiSeq chips that contained a synthetic oligonucleotide library encoding substitutions within the PAM and the target DNA sequence. DNA binding was imaged at eleven Cascade concentrations ranging from 63 pM to 630 nM (see Star Methods). At each concentration, the thermophilic Cascade complex was first incubated in the chip at 60° C. to promote DNA binding. Next, unbound complexes were flushed out of the chip, and DNA-bound Cascade was rapidly cooled to room temperature and labeled in situ with fluorescent anti-FLAG antibodies (FIGS. 1A and 2). The T. fusca Cascade complex included a triple FLAG epitope on the C-terminus of the Cas6 subunit. This epitope tag did not alter DNA binding by the T. fusca Cascade, as reported for the E, coli Cascade complex. Significant Cascade loss was not observed nor photobleaching during image collection (˜15 minutes per protein concentration). Apparent K_{d} values were determined by fitting the fluorescence intensities of each DNA cluster at the eleven Cascade concentrations to the Hill equation (FIG. 1D, Star Methods). Non-specific DNA binding was observed via a random DNA sequence that was also included in the chip. This negative control sequence had an apparent K_{d} that was lower than the highest measured concentration (FIG. 1D, dashed curve). These fits were used to define apparent binding affinity (ABA), the difference in apparent ΔG between the negative control sequence and a sequence of interest. Positive values indicate stronger binding, and negative values were discarded as non-specific DNA binding. DNA sequences with at least 5 unique fluorescent clusters were included in the analysis, which provided average error of approximately 0.2 k_{B}T for the apparent binding affinity (FIG. 4B). Approximately 16 million target DNA sequences were sequenced, giving complete coverage of all possible six-nucleotide PAM variants, as well as all single- and double-nucleotide substitutions along the entire target DNA (FIGS. 1E and 1F). Paired-end reads of linearly amplified synthetic oligonucleotide libraries were used to minimize biases and errors from library construction, synthesis, and sequencing (FIG. 4C). To avoid chip-specific biases, experiments were performed on two independent MiSeq chips, which recapitulated the measured ABAs (r=0.88) (FIG. 1G). This CHAMP dataset resulted in ˜36,000 unique DNA sequences with ABAs that were above the non-specific DNA binding threshold (FIG. 1H). With this dataset, efforts were made to define the principles guiding Cascade-DNA interactions.

(2) Quantitative Profiling of the Protospacer Adjacent Motif (PAM)

In all CRISPR-Cas systems, the PAM flanks target DNA that is complementary to the crRNA. The PAM is crucial for facilitating interrogation of the target DNA by the Cascade complex. Diverse PAMs can also bias CRISPR-Cas systems towards DNA degradation (interference) or spacer acquisition (adaptive immunity). Early studies proposed that Cascade recognizes a three nucleotide PAM. However, recent structural and sequencing studies of the E, coli Cascade complex suggested that Cse1 is sensitive to an extended PAM. Thus. CHAMP was used to determine the apparent binding affinity of Cascade towards six nucleotide PAMs when the target DNA is fully complementary to the corresponding crRNA.

CHAMP profiling of all 4,096 unique six nucleotide PAMs resulted in 950 sequences that had a non-zero ABA. In order visualize the complete set of all PAM preferences, sequence specificity landscapes (called PAM landscapes here) were adapted. The PAM landscape displays all PAM-dependent ABAs as a series of concentric rings. The highest-affinity sequence for the first three PAM positions (A₋₃A₋₂G₋₁) is included in the center of the concentric rings. This innermost dataset displays the ABAs for all 6-nucleotide PAM sequences that contain a perfect match to the highest affinity three-nucleotide “minimal” PAM (N₋₆N₋₅N₋₄A₋₃A₋₂G₋₁ for T. fusca Cascade: 64 unique sequences). The height and color of each bar on the individual rings corresponds to the ABA. A grey line above each peak represents the standard deviation of each measurement, as determined by bootstrap analysis. The vertical bars are sorted from the highest to lowest affinity sequences for each minimal PAM. When paired with AAG, variation in the −6 to −4 position contributes minimally to the ABA. The next ring in the landscape shows ABAs for six nucleotide PAMs that vary from A₋₃A₋₂G₋₁ by a single nucleotide in the first three positions (e.g., N₋₆N₋₅N₋₄C₋₃A₋₂G₋₁). The final ring shows PAMs that vary from A₋₃A₋₂G₋₁ by two nucleotides (e.g., N₋₆N₋₅N₋₄C₋₃C₋₂G₋₁). No measurable binding affinity to PAMs were detected with three substitutions relative to A₋₃A₋₂G₋₁. This representation gives a high-level overview of the entire PAM sequence space, reducing the high-dimensionality of CHAMP datasets for rapidly comparing the binding affinity to various PAMs.

The relative importance of each base was determined in the extended PAM by computing the maximum change in the ABA when only that base was varied. For example, a single data point in the violin plot for the PAM₋₂ position plots the maximum difference in ABAs for the four A₋₆A₋₅A₋₄A₋₃N₋₂A₋₁ PAMs. The violin plot extends this comparison for all possible PAMs at each of the six PAM positions and show the maximum effects of a single base change in varying PAM contexts. The PAM₋₂ position is the most critical for defining the highest-affinity T. fusca PAM. In contrast, the closely-related E, coli Cascade complex has promiscuous recognition at the PAM₋₂ position. Both PAM₋₁ and PAM₋₃ make similar contributions to the ABA. Subsequent positions in the extended PAM typically contribute less to ABA (PAM₋₂>PAM₋₁≈PAM₋₃>PAM₋₄>PAM₋₅>PAM₋₆). These results also highlight that PAMs with intermediate ABAs are the most sensitive to the identity of nucleotide positions −4 to −6. For example, for NNNGAG, the ABA increases over 60%, from 2.7 k_{B}T for GGAGAG to 4.4 k_{B}T for CACGAG. The data highlights additional sequence preferences, including enrichment of C₋₅ and G₋₆ in the highest affinity extended PAMs. The PAM₋₄ position is likely decoded by direct interactions with Cse1, as reported for the E, coli Cascade structure. Contributions of PAM₋₅ and PAM₋₆ can be due to indirect effects such as changes in the shape of the DNA minor groove.

The CHAMP results were compared with in vitro electrophoretic mobility shift assays (EMSAs) and in vivo interference assays. EMSAs showed excellent agreement with the CHAMP datasets (r=0.96) over three orders of magnitude in concentration. As expected, purified Cascade complexes lacking the Cse1 subunit did not exhibit any target DNA binding via EMSAs or CHAMP. Next, a plasmid-based interference assay was carried out and compared the results to those obtained via CHAMP for a variety of PAM sequences. In this assay. T. fusca Cascade, along with Cas3 nuclease, is induced in cells that also harbor a target plasmid that is degraded by the Cascade-Cas3 complex. After a brief outgrowth without antibiotics, interference efficiency is scored as the relative number of antibiotic-resistant colonies. The results showed a strong correlation (r=0.89), indicating that CHAMP-derived binding affinities are also predictive of interference activity in vivo. Moreover, the observations also help to explain how T. fusca avoids self-targeting its two Type I-E CRISPR loci. The first locus has a repeat that contains a 5′-A₋₄C₋₃C₋₂G₋₁ sequence adjacent to the CRISPR spacer elements, whereas the second repeat is 5′-T₋₄C₋₃A₋₂C₋₁. Herein is shown that these sequences strongly disfavor Cascade binding and thus limit auto-immunity at the CRISPR locus. In sum, CHAMP profiling recapitulates DNA binding affinities measured via EMSAs in vitro and is highly correlated with in vivo interference activity.

(3) Profiling Off-Target CRISPR-Cas DNA Binding on Synthetic DNA Libraries

To delineate the sequence determinants that influence Cascade-DNA interactions the ABA was analyzed for all DNA molecules with single or double substitutions along a 35-nt region that includes the first three positions of the PAM and the target DNA (FIG. 5). CHAMP profiling yielded information for all possible single-base substitutions with an average 3,000-fold coverage (FIG. 5A). As expected, substitutions in the PAM region reduced the ABA substantially, with the second position being most critical for Cascade binding (FIG. 5A). Prior structural and biochemical studies have established that every sixth nucleotide is not paired with the crRNA and flipped out in the Type I-E Cascade-DNA complex. A clear signature for these flipped-out base positions is also evident in the CHAMP profiling data (FIG. 5A). Surprisingly. CHAMP revealed that Cascade affinity was increased when thymidine replaced the complimentary cytosine as the third flipped-out base (position 18). A preference for thymidines over cytosines at the flipped out positions was confirmed via EMSA assays. In line with these observations, a structural study proposed that flipped out bases interact with a molecular relay of Cse2-encoded arginines. Taken together, these results indicate that flipped-out and mismatched DNA bases likely interact with Cascade, further stabilizing partially mismatched crRNA-DNA complexes during both interference and primed acquisition.

A simple model was developed to better quantify how substitutions along the PAM and the target DNA affect Cascade binding (FIGS. 5B-D). This model considers a position-dependent penalty for all single base substitutions (FIG. 5C) and a position-independent weight that accounts for the identities of each target and substituted base (FIG. 9D). This model has fewer parameters than position weight matrices, but nonetheless described ˜90% of the variance in the experimental data (FIG. 5B). To further constrain this model, a second CHAMP dataset with a second crRNA-Cascade complex targeting a different DNA sequence was acquired. The model accurately described both independent CHAMP datasets acquired with two different crRNAs and corresponding DNA libraries (r=0.92) (FIG. 5B). Analysis of the position-specific penalties clearly highlights the importance of the PAM, as well as the PAM-proximal nucleotides (i.e. seed region) in modulating the affinity of Cascade for DNA. The overall substitution penalties decrease with increasing distance from the PAM (FIG. 5C). This pattern has been recently observed for other CRISPR-Cas systems, and likely reflects the initiation and directional formation of an R-loop proceeding from the seed region.

The ABAs were analyzed for all double nucleotide substitutions along the same 35-nt PAM and target DNA region (FIG. 5E). The data highlights the importance of the PAM₋₂ position for controlling Cascade binding, as well as the synergistic effects of having any two flipped out bases. In the seed region, single substitutions are already poorly tolerated and reduce ABAs significantly. Therefore, a second mismatch in the seed reduces the ABAs to DNA-binding levels that are like non-specific DNA, while a second mismatch in PAM-distal positions are often tolerated. Two substitutions in the PAM-distal sequence only marginally destabilized the Cascade-DNA complex.

Surprisingly, the data and model also reveal an additional periodicity in base-substitution penalties centered between the flipped-out bases (FIGS. 5C and 5E). This periodicity results in an overall decrease in mismatch penalties every three nucleotides (e.g., at +3, +6, +9, etc.). A close inspection of the high-resolution E, coli Cascade structure reveals that every third base pair is puckered due to steric clashes between the RNA-DNA duplex and several residues in the Cas7 subunit (FIGS. 5F and 5G). Six repeats of the Cas7 subunits polymerize along the crRNA to form the backbone of the Cascade complex. These subunits give rise to the three-nucleotide periodicity observed herein and dinucleotide ABA data. Moreover, these residues are highly conserved amongst divergent Type I-E CRISPR-Cas systems, indicating that they play a role in Cascade assembly. Overall, the results highlight an unanticipated three-nucleotide periodicity in Cascade-DNA binding penalties that reduce the overall fidelity of RNA-DNA binding.

(4) Profiling Off-Target CRISPR-Cas Binding in Human Genomic DNA

CHAMP uses a standard Illumina workflow and is immediately compatible with any nucleic acid library, including those derived from genomic preparations. CHAMP was extended to profile CRISPR-Cas binding on human genomic DNA (FIG. 6). To enrich for gene-coding regions, exome capture was used in conjunction with paired-end sequencing on an Illumina MiSeq sequencer (FIG. 6A). The resulting sequenced MiSeq chip had an average 11-fold coverage for 17,862 human protein-coding regions from 7 million unique high-quality DNA clusters (FIG. 7A). This MiSeq chip was used to quantitatively assay off-target CRISPR-Cas binding. Remarkably, 37 genes showed at least one high-affinity CRISPR binding site (defined as ABAs>4 k_{B}T) and ˜200 genes showed moderate-affinity ABAs (>3 k_{B}T). The precision of the off-target DNA sequence is defined by both the length distribution of the sheared exome fragments and the depth of coverage at each position (FIG. 6B). Nonetheless, most genes harboring off-target sites showed a single, well-resolved ˜200 bp-wide peak (FIG. 6C).

The peaks with the highest ABAs represent genomic high-affinity off-target DNA binding sites. A subset of these peaks represent a combination of two lower affinity binding sites that are closer than the nominal resolution of 210 bp (FIG. 7B). Nonetheless, a logo analysis of all peaks with ABAs>3 k_{B}T revealed a consensus sequence that matches closely with the expected critical determinants of off-target binding observed in the synthetic DNA libraries (FIG. 6D). The consensus off-target site had a strong preference for an AAG PAM, with the second adenine giving the strongest signal. Second, off-target sites were highly enriched for the first eight basepairs of the target DNA sequence. One notable exception is the flipped-out base in the sixth position, which does not base pair with the crRNA (also see FIG. 9). Consistent with binding data obtained from synthetic DNA arrays (FIG. 9), mismatches are also tolerated at the third base, which has reduced base pairing with the crRNA. This data also highlights that a six-nucleotide PAM-proximal “seed” region can be important for efficient binding. Herein it was demonstrated that CHAMP can profile off-target CRISPR-Cas binding sites in human genomic DNA, paving the way for rapid and quantitative profiling of off-target binding sites in patient-specific genomes.

(5) Cas3 Recruitment Requires Perfect Base Pairing Near the PAM

CHAMP profiling revealed pervasive off-target DNA binding by Cascade. It was reasoned that subsequent binding of the Cas3 nuclease constitutes an additional sequence-dependent proofreading mechanism. This possibility was investigated with three-color CHAMP experiments that measured the degree of Cas3 recruitment to DNA-bound Cascade (FIG. 8A). Fluorescent Cascade, Cas3, and alignment markers were spectrally separated into three distinct emission channels. After adding alignment markers. Cascade was introduced into the chips at a sufficiently high concentration to bind most DNA clusters that were partially complementary to the crRNA. Next, a saturating concentration of Cas3 was introduced into the same chip and CHAMP data was acquired (FIG. 8B). To prevent Cas3-dependent DNA degradation, these assays were conducted with a buffer containing 1 mM AMP-PNP and lacking Co⁺² (see Star Methods). While most clusters had a linear correlation between Cascade and Cas3 signals, a subset of the clusters deviated from this linear correlation with a reduced Cas3 fluorescence (FIG. 8B, inset). As expected, no Cas3 binding to the DNA clusters was observed when Cascade was omitted from the chip, or on clusters that did not bind Cascade. These results indicate that Cas3 is recruited to Cascade in a DNA sequence-dependent manner.

Approximately, 646,000 DNA clusters representing 10,810 unique DNA sequences were analyzed to determine the requirements for efficient Cas3 recruitment. This dataset represented all extended PAM and single-nucleotide substitution variants, as well as 94% of double-nucleotide substitution variants along the target DNA sequence (FIG. 1F). Approximately 450 DNA sequences showed a reduced ratio of Cas3 to Cascade fluorescent intensities relative to that of the fully complementary DNA target sequence. To better understand why Cas3 was not recruited at the same level to all DNA clusters, focus was turned to DNA sequences with single nucleotide substitutions along the PAM and the target DNA (FIG. 8C). Comparing the Cas3 and Cascade fluorescent signals indicated that most DNA sequences fell on a diagonal line that indicates stoichiometric Cas3 recruitment, while those below the diagonal line indicate sub-stoichiometric Cas3 to Cascade ratios. As expected, no points were observed above the diagonal (FIG. 8C). Cas3 recruitment was partially compromised at nearly all non-AAG PAMs, as well as for target DNAs with a substitution in the first three PAM-proximal positions (FIG. 8C). Using this information, it was determined how sequence-dependent substitutions in the target DNA impact Cas3 recruitment. These results are expressed as a Cas3 recruitment penalty relative to expected stoichiometric binding (FIG. 8D). Surprisingly, the results revealed that mismatches in PAM₋₁ and +1 target positions strongly compromised Cas3 recruitment (FIG. 8D). These data implicate the PAM, as well as the first few nucleotides in the seed region, as critical for Cas3 binding to a Cascade-DNA complex.

(a) Sequence-Specific Loss of Cse1 Decreases the Cascade Interference Efficiency

EMSAs and nuclease assays were used to further determine the mechanism of DNA-guided Cas3 recruitment. Cascade readily binds target DNA containing an A₋₃A₋₂G₋₁ PAM. Surprisingly, the Cascade-DNA complex migrated as a faster mobility species when either this PAM was changed or when the +1 DNA position was mismatched relative to the crRNA. Indeed, a DNA:crRNA mismatch in the +1 position converted 80% of the Cascade complexes to the faster-migrating species. These effects were additive, as changing the PAM and the +1 position simultaneously resulted in nearly 100% of the faster-migrating sub-complex. It was confirmed that this faster migrating species represents Cascade lacking the Cse1 subunit. Adding a large excess of free Cse1 can restore the mobility back to that of a complete Cascade complex. Cse1 physically interacts with Cas3 and loads the nuclease onto the target DNA. Adding excess Cas3 resulted in a super-shift, but only when Cse1 was part of the Cascade complex. As expected, impaired Cas3 recruitment also reduced Cas3 nuclease activity when ATP and Co⁺² were added to the reaction mixtures. Consistent with these in vitro studies, disrupting either the PAM or first few seed nucleotides also caused strong reduction in the plasmid-based in vivo interference assays. These results reveal that DNA sequence-specific loss of Cse1 abrogates Cas3 recruitment and provides an additional proofreading mechanism for modulating CRISPR interference.

b) Discussion

CHAMP repurposes sequenced and discarded chips from modern next-generation Illumina sequencers for high-throughput association profiling of proteins to nucleic acids. A key difference between CHAMP and prior NGS-based approaches is that it does not require any hardware or software modifications to discontinued Illumina sequencers. In CHAMP, all association-profiling experiments are carried out on sequenced MiSeq chips and imaged in a conventional TIRF microscope. CHAMP's computational strategy uses phiX clusters as alignment markers to align the spatial information obtained via Illumina sequencing with the fluorescent association profiling experiments. This strategy offers three key advantages over previous approaches. First, using a conventional fluorescence microscope opens new experimental configurations, including multi-color co-localization and time-dependent kinetic experiments. The excitation and emission optics can also be readily adapted for FRET (see FIGS. 9A, 9B and 9C), and other advanced imaging modalities. Second, complete fluidic access to the chip allows addition of other protein components during a biochemical reaction. Third, the computational strategy for aligning sequencer outputs to fluorescent datasets is applicable to all modern Illumina sequencers, including the MiSeq, NextSeq, and HiSeq platforms. Indeed, the CHAMP imaging and bioinformatics pipeline was also used to regenerate, image, and spatially align the DNA clusters in a HiSeq flowcell (FIGS. 9D, 9E, 9F and 9G), providing an avenue for massively parallel profiling of protein-nucleic acid interactions on both synthetic libraries and entire genomes. On-chip transcription and translation (e.g., ribosome display) can be leveraged to facilitate high-throughput studies of RNA or peptide association landscapes. These studies permit quantitative biophysical studies of diverse protein-nucleic acid interactions.

(1) Cascade Interrogates an Extended PAM and Recognizes Mismatched DNA Targets

Using CHAMP, the biophysical properties governing interactions between target DNA and the Type I-E CRISPR-Cas effector complex were profiled. The findings reveal the biophysical parameters governing PAM recognition and DNA-binding at partially-complementary target DNAs. T. fusca Cascade first identifies an extended PAM, possibly via hydrogen bonds with the PAM₋₄ nucleotide as indicated by a recent high-resolution structure of the E, coli Cascade-DNA complex. Further readout of the PAM₋₅ and PAM₋₆ positions can be mediated by indirect effects, such as changes in the major and minor groove widths at the PAM-proximal bases. These results are also broadly consistent with recent plasmid-based PAM-profiling experiments, which highlighted that diverse CRISPR-Cas systems—including the E, coli Type I-E Cascade—all decode an extended PAM.

Following PAM recognition and target DNA unwinding, an R-loop extends along the complementary target DNA. Using CHAMP, the effects of multiple sequence substitutions on Cascade-DNA interactions were probed. In addition to identifying the importance of the PAM, “seed,” and flipped-out bases, the analysis and modeling revealed an unanticipated three-nucleotide periodic interaction that reduced the relative penalty for DNA-RNA mismatches at these positions. A re-analysis of previously reported E, coli Cascade plasmid interference assays also shows the same three-nucleotide periodicity. This is a general structural feature shared by other Type I-E systems and that it arises due to a steric clash between basepairs in the R-loop and residues in each of the six Cas7 subunits. The crRNA is required for assembly of the E. coli Cascade complex, and these periodic contacts allow the crRNA to act as a scaffold during Cascade assembly. The crRNA is held in a conformation that maximizes interaction with the target DNA, possibly avoiding secondary structure formation by targets, as has been demonstrated in other RNA-guided nucleases. This periodic mismatch tolerance was also confirmed at off-target sites mapped to the human exome, further highlighting the importance of quantitatively mapping the influence of mismatches on CRISPR-DNA interactions with both synthetic and genomic DNA substrates.

(2) A DNA Sequence-Dependent Mechanism Underlies Cse1 Loss and CRISPR Interference

By performing multi-color CHAMP imaging, is was discovered Cas3 recruitment is dependent on the identity of the PAM, as well as perfect complementarity between crRNA and DNA in the +1 to +3 positions. These nucleotides interact with the Cse1 subunit of the Cascade complex. EMSAs and in vitro nuclease assays revealed that T. fusca Cse1 dissociates from Cascade at intermediate PAMs or when there are mismatches between the crRNA and the first three nucleotides of the target DNA. The functional significance of this position was further confirmed with in vivo plasmid interference assays and also recapitulates previously published in vivo interference results with the E, coli Cascade complex.

In addition to identifying foreign DNAs, Cascade and Cas3 also promote primed spacer acquisition, where additional spacers are rapidly acquired from foreign DNAs that already contain a spacer in the CRISPR locus. Spacer acquisition requires the Cas1-Cas2 protein complex, which binds protospacer DNA and uses its integrase activity to insert the protospacer within the CRISPR array. Cascade can promote target acquisition at both perfectly matched spacers and mismatch-containing spacers that do not elicit strong interference. Conformational control of the Cse1 subunit is emerging as a key paradigm for recruiting Cas1-Cas2 and redirecting the Cascade-Cas3 complex towards primed acquisition. Herein is shown that Cse1 undergoes a DNA-sequence dependent conformational change that renders it labile in the absence of Cas1-Cas2 complex.

(3) Leveraging CHAMP for Mapping Protein-Nucleic Acid Interactions on Human Genomes

Because CHAMP uses the standard Illumina workflow, it is immediately compatible with any nucleic acid library, including synthetic DNA, RNA, or genomic preparations. However, mapping CRISPR-DNA interactions on sequenced genomes presents additional computational challenges due to the random shearing lengths and uneven sequencing coverage. To address this challenge, a bioinformatics pipeline was developed that successfully identified off-target binding sites within a human exome with a ˜200 bp effective resolution at an average 11-fold coverage depth. Higher resolution mapping can be readily achieved by shorter DNA fragments and greater sequencing coverage. Thus, CHAMP can be used to probe off-target CRISPR-Cas binding in any genome prior to performing genome-editing. Extensions allow for direct observation of both binding and cleavage at these off-target sites. As CRISPR-Cas systems continue to be developed for human gene modification, CHAMP and similar methods are useful tools for rapidly and quantitatively assaying target specificity on individual patient's genomes.

The chip hybridized association-mapping platform (CHAMP) described in this study adds to a growing toolbox of high-throughput methods for determining aspects of protein-DNA interactions. These methods can be broadly classified by the information content (from hundreds to millions of unique interactions probed in parallel), the types of DNA sequences that can be interrogated (e.g., synthetic oligonucleotides and/or genomic libraries), and the detection schemes used to infer biophysical parameters. CHAMP differs from most of these methods because all profiling experiments are carried out on used MiSeq or HiSeq chips that are generated during the Illumina-based next generation DNA sequencing workflow. Current MiSeq chips generate up to 25 million unique DNA clusters, and the HiSeq generates up to 10 billion unique DNA clusters, and both are compatible with synthetic and genomic DNA libraries. Proteins are fluorescently labeled and a conventional fluorescence microscope is used to image protein binding to each DNA cluster. Using a fluorescence microscope opens new experimental configurations, including multi-color co-localization, time-dependent kinetic experiments. FRET, and other advanced imaging modalities.

Surface plasmon resonance (SPR) is a label-free imaging modality that can directly measure binding constants between proteins and synthetic nucleic acids. Most commercial SPR instruments are limited to measuring a single protein-nucleic acid interaction per experiment. More recently, several groups have adapted SPR and other label-free imaging modalities for multiplexed data acquisition. The parallel acquisition of 120 unique DNA sequences with a single protein has also been reported and SPR microscopes that can accommodate hundreds of spots have been developed. While SPR can independently measure both on and off rates, it remains a relatively-low throughput method. Multiplexed SPR studies are not yet able measure DNA-sequence specific multi-protein complex assembly.

Systematic evolution of ligands by exponential enrichment (SELEX) is a well-established technique for finding sequences preferred by a DNA-binding protein. For SELEX, a synthetic or genomic DNA library is incubated with immobilized protein. The protein is then washed to remove unbound DNA, the protein-bound DNA is eluted, PCR amplified, and sequenced. The cycle is repeated with the bound DNA from each round of selection with increasingly more stringent washes. A high-throughput SELEX variant permits the analysis of several affinity-tagged proteins in parallel followed by multiplexed sequencing. While SELEX can determine the highest affinity DNA sequences, it does not determine kinetic parameters. SELEX is also less appropriate for determining biophysical mechanisms because it removes weakly-binding species during subsequent washing cycles.

Several conceptually related methods (e.g., ChIP-Seq, Bind-n-Seq and Spec-Seq) use next generation DNA sequencing to measure the enrichment of protein-bound DNA sequences in either genomic or complex synthetic DNA libraries. In these methods, the DNA library is incubated with a DNA (or RNA)-binding protein. When the binding reaction reaches equilibrium (or is crosslinked in cells for ChIP-Seq), the bound protein-DNA complexes are separated from free DNA. Proteins can be selectively purified using an immobilized antibody (as in ChIP-Seq) or by native gel separation and DNA extraction. Protein-bound DNA is then sequenced and a sequence logo can then be calculated using existing software. These methods are conceptually simple, label-free, and can be very high-throughput owing to the sequencing-based readout of protein binding. However, the quality of data is dependent on the ability to selective enrich for the desired protein-DNA complexes. For ChIP-Seq, the antibody quality is especially important. Bind-n-Seq requires gel fractionation that can disrupt transient or weak interactions. Measuring multi-protein interactions also requires that gel electrophoresis be used to separate all possible DNA-bound species. Finally, these methods cannot directly measure other biophysical parameters, such as off-rates and conformational transitions (e.g., via FRET).

Microfluidic systems have been built to assay hundreds or thousands of protein-DNA interactions in parallel. Maerkl and Quake developed a system that combines microfluidic channels with a DNA microarray, effectively creating thousands of isolated reaction chambers. Fluorescently-labelled DNA with a variety of sequences and concentrations is spotted into different chambers, each containing a surface-bound protein of interest. After a period of incubation, bound protein-DNA complexes are mechanically immobilized while unbound DNA is washed away. The fluorescence of the DNA is measured, which can then be used to determine the affinity for each sequence. Ultimately, almost five hundred DNA sequences at various concentrations were analyzed. A similar technique was used to study the affinity of transcription factors to either 32 or 128 unique sequences over 32 concentrations. One advantage of these systems is that the bound DNA can be locked in place by mechanical force, effectively “freezing” the signal at equilibrium. However, these systems remain limited to a few thousand reaction chambers, require complex microfabrication expertise, and cannot readily measure binding affinities to genomic DNA samples where the DNA sequence in not known a priori.

Protein-binding microarrays (PBMs) contain tens of thousands of spots of heterogeneous DNA with known sequences. To measure the strength of sequence-specific protein-DNA interactions, fluorescently-labeled proteins are flowed onto the microarray, and the fluorescence intensity of each spot is measured. As such, PBMs are some of the earliest instantiations of high-throughput surface-tethered protein-nucleic acid interaction platforms. By using synthetic oligonucleotides, PBMs can represent all possible eight-mer DNA sequences with good statistical coverage. The signals can then be analyzed to determine the strength of each interaction, ultimately leading to a sequence logo. While this approach is higher throughput than SPR, being limited to eight-mers makes PBMs unusable for studying CRISPR nucleases or proteins with larger DNA-binding footprints.

A series of related methods (e.g., HiTS-FLIP, HiTS-RAP, RNA-MaP) extended PBMs to directly measure protein-nucleic acid interactions on modified Genome Analyzer II DNA sequencers. First, an unmodified Genome Analyzer instrument is used to sequence the DNA. The resulting chip is then loaded into a second, user-modified Genome Analyzer with upgraded imaging hardware and custom-written control software. For profiling RNA interactions, the DNA clusters are transcribed on-chip. Afterwards, a fluorescently-labeled protein is flowed onto the chip containing the sequenced DNA, and the fluorescent intensity of each DNA sequence is then measured. By observing multiple concentrations, sequence-specific binding affinities can be determined for hundreds of thousands of unique DNA sequences. The primary drawback of these methods is that they are locked to a single sequencer that requires significant user upgrades. This sequencer—the Genome Analyzer II—is no longer sold or supported by Illumina. HiTS-FLIP has also only been demonstrated to work with a single fluorescent protein, likely due to the limitations associated with the Genome Analyzer hardware. CHAMP significantly expands these methods because it is compatible with all modern sequencers, does not require any modifications to the sequencer hardware, and can be used to measure additional biophysical parameters such as multi-protein interactions. Use of three independent fluorescent colors is already supported by the software and is demonstrated in this manuscript. Most importantly, the associated bioinformatics pipeline can analyze binding to both synthetic DNA libraries and sheared genomic DNA. In sum, CHAMP substantially improves existing high-throughput methods for profiling protein-nucleic acid interactions.

c) Star*Methods

(1) Protein Cloning and Purification

T. fusca Cascade and Cas3 were over-expressed and purified. Briefly, the Cascade complex and crRNA were expressed from pET-based plasmids that were co-transformed into BL21 star (DE3) cells (Thermo-Fisher). Cse1 contained a His₆/Twin-Strep/SUMO N-terminal fusion, while Cas6 contained an N-terminal triple FLAG epitope for fluorescent labeling. Single colonies were used to inoculate LB+Kanamycin/Carbenicillin/Streptomycin media. At OD₆₀₀ 0.8, cells were induced with 1 mM IPTG overnight at 25° C. Cells were then lysed in 20 mM HEPES, pH 7.5, 500 mM NaCl, 2 μg mL⁻¹ DNase (GoldBio) and 1×HALT protease inhibitor (Thermo-Fisher), and the clarified lysate was applied to a hand-packed Strep-Tactin Superflow gravity column (IBA Life Sciences) for purification via the Twin-Strep tagged Cse1. The Cascade complex was eluted with 20 mM HEPES, pH 7.5, 500 mM NaCl, 5 mM desthiobiotin, and then concentrated by centrifugal filtration (30 kDa Amicon, Millipore). The concentrate was then incubated overnight at 4° C., with 3.3 μM SUMO protease to remove tags from Cse1. The complex was further fractionated over a HiLoad 16/600 Superdex 200 column (GE Healthcare) equilibrated in storage buffer (10 mM Tris-HCl, pH 7.5, 150 mM NaCl, 5 mM DTT). Fractions containing the full Cascade complex were determined by SDS-PAGE, pooled, and concentrated to ˜5-10 μM (30 kDa centrifuge concentrators, Millipore). Small aliquots were flash frozen in liquid nitrogen and stored at −80° C. Aliquots were used only once and not refrozen.

(2) Antibodies

Cascade and Cas3 were fluorescently labeled with mouse anti-FLAG M2 (F3165, Sigma) and Rabbit anti-HA (RHGT-45A-Z, ICL labs), respectively. Antibodies were conjugated to Alexa488 or Alexa647 at a ratio of ˜1:3 antibody:dye according to the manufacturer's instructions (Molecular Probes Alexa Fluor antibody labeling kits, Thermo Fisher Scientific). The antibody to dye conjugation ratio was measured using a NanoDrop (Thermo Fisher Scientific) according to the manufacturer-provided protocol. Fluorescent antibodies were stored in PBS buffer (pH 7.2, with 2 mM sodium azide) at −20° C.

(3) DNA Oligonucleotides Libraries

Oligonucleotides were purchased from IDT or IBA (see Table 3).

TABLE 3

| ligo. | | |
|---|---|---|
| ID | [symbol] | Sequence |
| J.TA-C | [symbol] | AATGATACGGCGACCACCGAGATCTACACTCTTTCCCTACACGACGCTCT |
| | | TCCGATCTAAGGCCGAATTCTCACCGGCCCCAAGGTATTCAAGAGATCGGAAGA |
| | | GCACACGTCTGAACTCCAGTCACTTGTTCTTTTGCACTACCGTCAGGTAATCTCG |
| | | TATGCCGTCTTCTGCTTG (SEQ ID NO: 1) |
| J.TAflipped T | [symbol] | 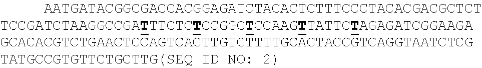 |
| J.TAflipped C | [symbol] | 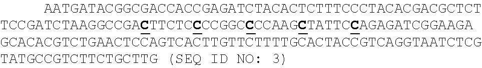 |
| J.TA-7NSeed | [symbol] | 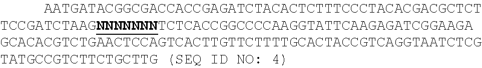 |
| J.TADopedT | [symbol] | 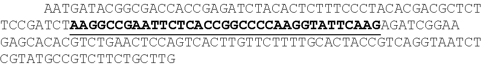 |
| J.TBDopedT | [symbol] | 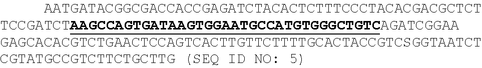 |
| J.RP | [symbol] | GTGACTGGAGTTCAGACGTGT (SEQ ID NO: 6) |
| J.atto6 47-PCP | [symbol] | Atto647/CGGTCTCGGCATTCCTGCTGAACC (SEQ ID NO: 7) |
| J.Cy3-PCP | [symbol] | Cy3/CGGTCTCGGCATTCCTGCTGAACC (SEQ ID NO: 8) |
| J.P5 | [symbol] | AATGATACGGCGACCACCGAGA (SEQ ID NO: 9) |
| J.Cy5-P5 | [symbol] | Cy5/AATGATACGGCGACCACCGAGA (SEQ ID NO: 10) |

*Randomized sequences are underlined and bold. [symbol]
indicates data missing or illegible when filed

A synthetic oligonucleotide with six randomized bases was purchased from IDT and used to profile the extended six nucleotide PAM. Two additional synthetic oligonucleotide libraries were designed to measure the effects of mismatches along the entire target DNA sequence. These libraries were made by randomizing the bases along the entire length of the consensus target DNA sequence. In these “doped” libraries, every correct base had a 9% change of being substituted for each of three other bases (3% each; 9% total). This doping mixture was chosen to provide comprehensive coverage for sequence variants with a Hamming distance less than three on a typical MiSeq chip (representing ˜20-25 million unique reads). Pooled custom DNA libraries were also purchased from CustomArray. DNA libraries were sequenced on a MiSeq (Illumina) using a 2×75 or a 2×300 paired end reagent kit (v3).

(a) Exome Preparation and Sequencing

HeLa genomic DNA (NEB N4006S) was prepared using the TruSeq Exome Library Prep Kit (Illumina), yielding approximately 170 basepair-long DNA fragments. The exome library was then sequenced using the MiSeq Reagent Kit v3 (Illumina, 2×300 paired-end reads). The resulting MiSeq run yielded 9.1 million exome reads.

(4) Chip Regeneration and Addition of Alignment Markers

After sequencing, MiSeq chips were kept at 4° C. in storage buffer (10 mM Tris-Cl, pH 8.0, 1 mM EDTA, 500 mM NaCl). All imaging and chip regeneration steps were carried out in a custom-built microscope stage adapter with integrated microfluidic interconnects. An overview of the microscope stage and fluidic interface is summarized in FIG. 2. Detailed blueprints of all components are also available via GitHub. Temperature was controlled by PiWarmer, a home-built Raspberry Pi-controlled heating element. PiWarmer was also used to run the heating and cooling cycles required for on-chip cluster regeneration. Schematics and code for assembling the temperature controller, as well as protocols for chip regeneration are available via GitHub. The heating element was mounted on the microscope turret to allow for easy and consistent heat application.

All fluidic methods utilized an automated syringe pump (KD scientific) operating at a flow rate of 100 μl min⁻¹ for chip preparation and experimentation. All reagents were added to the flow path through an automated, multi-position valve (Rheodyne MXP9900) containing either a 100 or 700 μL injection loop.

To regenerate the DNA clusters, all DNAs covalently affixed to the MiSeq chip surface were denatured with 500 μl 0.1 N NaOH as it flowed through the chip (5 minutes) and similarly washed with 500 μl TE buffer. This removed the untethered DNAs strands containing residual fluorescent dyes from sequencing (see FIG. 2). After denaturation, the chip was heated to 85° C., and incubated with 500 nM of the regeneration primer (CJ.RP) in hybridization buffer (75 mM Trisodium Citrate, pH 7.0, 750 mM NaCl, 0.1% Tween-20). CJ.RP was annealed at 85° C., for 5 min, followed by ramped linear cooling to 65° C. over 10 min, ramped linear cooling from 65° C. to 40° C. over 30 min, and then washed with 1 ml washing buffer (4.5 mM Trisodium Citrate, pH 7.0, 45 mM NaCl, 0.1% Tween-20) at 40° C. (10 minutes). CJ.RP binds to all user clusters but does not target phiX clusters. CJ.RP was extended at 60′C for 10 minutes in isothermal amplification buffer (20 mM Tris-HCl, pH 8.8, 10 mM (NH₄)₂SO₄, 50 mM KCl, 2 mM MgSO₄, 0.1% Tween-20) containing 0.08 U/μl of Bst 2.0 WarmStart DNA polymerase (New England Biolabs) and 0.8 mM of dNTPs. The chip was then washed with 500 μl hybridization buffer at 60° C. to remove the polymerase (5 minutes). Finally, a phiX primer labeled with Atto647 or Cy3 (atto647-PCP/Cy3-PCP) was annealed under the same conditions as CJ.RP. The resultant fluorescent phiX clusters were used for aligning the FASTQ points to imaged clusters (see FIG. 2 and Star Methods below). Prepared chips can be used for at least a dozen Cascade-DNA binding experiments before requiring regeneration.

(5) Fluorescence Microscopy

All fluorescence images were collected using a Nikon Ti-E microscope in a prism-TIRF configuration equipped with a motorized stage (Prior ProScan II H117) containing the experimental MiSeq chip (Illumina) housed in a custom stage adapter (FIG. 2). The chip was illuminated with 488 nm (Coherent), 532 nm (Ultralasers), or 633 nm (Ultralasers) lasers through a quartz prism (Tower Optical Co.). The laser exposure was controlled with high-speed shutters (LS682Z0, Vincent Associates) To minimize spatial drift, the microscope was assembled on a floating optical table (TMC). An active feedback system was used to maintain focus across the entire chip surface (Nikon PerfectFocus). Data were collected with a 100 ms exposure through a 60× water-immersion objective (1.2NA, Nikon) paired with (i) a quad-band filter (89401 Chroma), a 638 nm dichroic beam splitter, and either a 600) nm long-pass filter or 500 nm long pass/600 nm short pass filters (Chroma), or (ii) a dual-band filter (ZET532/660m Chroma), a 640 nm dichroic beam splitter, and either a 655 nm long-pass filter or ET4585/65m band pass filter (Chroma), which allowed multi-channel detection through two EMCCD cameras (Andor iXon DU897, cooled to −80° C.). Images were collected using Micro-Manager Open Source Microscopy software and saved in an uncompressed TIFF file format for later analysis via a custom written image-processing pipeline (see below).

(6) CHAMP Assays

Increasing concentrations of the Cascade complex (0.063, 0.16, 0.39, 1, 2.5, 6.3, 16, 39, 100, 250, and 630 nM) were injected into a regenerated MiSeq chip and incubated at 60° C., for 10 min in imaging buffer (40 mM Tris-HCl, pH 8.0, 150 mM NaCl, 2 mM MgCl₂, 1 mM DTT, 0.2 mg ml⁻¹ BSA, 0.1% Tween-20). After the incubation, excess Cascade was rapidly flushed out of the chip while the remaining proteins were labeled; this was accomplished by washing with 100 μl imaging buffer at 60° C., then 100 μl of 20 nM fluorescently-conjugated anti-FLAG antibody in imaging buffer at 25° C., and then an additional 100 μl of imaging buffer at 25° C. (3 minutes total). Control experiments that omitted Cascade indicated that the fluorescent antibodies did not bind to the chip surface.

For each Cascade concentration, up to 812 fields of view were imaged spanning nearly 50% of the total sequenced MiSeq chip surface area. The chip was illuminated with 20, 40 or 30 mW of laser power at 488, 532, or 633 nm, respectively (measured at the front face of the TIRF prism). To prevent photobleaching, the lasers were shuttered between subsequent fields of view during the ˜15 minutes of image acquisition. No appreciable Cascade dissociation or cluster photobleaching occurred during this time. In order to avoid pixel saturation at high protein concentrations, ten 100 ms images were captured at each field of view. These images were summed into a final image and stored in hdf5 file format by channel and position. Care was taken to minimize experiment-to-experiment variation by acquiring all concentrations of a titration series in a single day. Following each experiment, the MiSeq chips were deproteinized with 32 units of Proteinase K (New England Biolabs) in washing buffer for 30 minutes at 42° C., and the chip showed no sign of degradation even after twelve Proteinase K treatments. The DNA in a chip can be denatured and re-synthesized up to five times using the regeneration protocol described above.

(7) Electrophoretic Mobility Shift Assay (EMSA)

All EMSAs were performed with radioactively or fluorescently labeled PCR products containing the indicated PAM and protospacer, as well as flanking sequences used in the CHAMP experiments (i.e., Illumina adapters). PCR was performed using 1 ng of template plasmid containing the desired PAM/protospacer, 500 nM of P5 primer for radioactive-labeling or Cy5-P5 primer for fluorescent-labeling, 500 nM of CJ.RP, 200 μM of dNTPs and 0.5 unit of Q5 high-fidelity DNA polymerase (New England Biolabs) in a 25 μl reaction on an MJ Research PTC-200 Thermal Cycler. The PCR product was purified (PCR purification kit, Qiagen) and quantified on a Nanodrop spectrophotometer (Thermo Fisher Scientific). For radioactive assays, PCR products were labeled with γ³²P-ATP (PerkinElmer) using T4 polynucleotide kinase (New England Biolabs). The labeled PCR products were purified with MicroSpin G-25 columns (GE Healthcare).

Cascade binding assays were performed by incubating 0.1 nM of ³²P-labeled dsDNA with increasing Cascade concentrations (0.025, 0.063, 0.16, 0.39, 1, 2.5, 6.3, 16, 39, 100, 250, 630 nM) for 30 min at 62° C. in binding buffer (40 mM Tris-HCl, pH 8.0, 150 mM NaCl, 2 mM MgCl₂, 1 mM DTT, 0.2 mg ml-1 BSA, 0.01% Tween-20). The reactions were resolved on a 2.5% agarose gel run with 0.5×TBE buffer. Gels were dried and DNA was visualized using a Typhoon scanner (GE Healthcare). ImageQuant software (GE Healthcare) was used to quantify the bound and unbound DNA amounts. The fraction of bound DNA was fit to the Hill equation to obtain K_{d} values. All experiments were repeated in triplicate.

To observe Cas3 binding, Cascade (39 nM) and target dsDNA (2 nM) were pre-bound for 30 min at 62° C. in a binding buffer. Then, Cas3 and AMP-PNP (Sigma) were added into the EMSA reaction for final concentrations of 1.1 μM and 2 mM, respectively and incubated for 10 min at 62° C. The reactions were resolved on a 5% native PAGE gel containing 0.5×TBE buffer and visualized using a Typhoon scanner (GE Healthcare).

(8) Cas3 Nuclease Assays

Cascade (39 nM) was first incubated with Cy5-labeled target dsDNA (2 nM) for 30 min at 62° C. in binding buffer. Then, Cas3, CoCl₂ (Sigma) and ATP (Sigma) were added into the EMSA reaction at final concentrations of 650 nM, 111 μM and 1.9 mM, respectively and incubated for 30 min at 62° C. The reaction was quenched with 50 mM EDTA and deproteinized with proteinase K. The reactions were resolved on a 10% denaturing PAGE gel containing 0.5×TBE buffer and visualized using a Typhoon scanner (GE Healthcare).

(9) Plasmid Loss Assays

The Cascade expression construct was generated by insertion of the Cascade gene cassette (encoding all protein subunits) into a pBAD (ApR) vector. The pre-crRNA expression cassette containing five identical CRISPR units for target A, was cloned into the pACYC-Duet-1 (CmR) vector. A 127-bp fragment containing a protospacer and a PAM for target A was cloned into the pCDF-Duet-1 (SmR) vector to serve as the target DNA. In vivo assays were performed with T. fusca Cascade and Cas3.

(10) Computational Methods

The main challenge for CHAMP is the precise mapping of each individual DNA cluster to an underlying DNA sequence. This is because CHAMP uses images obtained via conventional TIRF microscopy and the information in these images is only partially encoded in the sequencing output generated by all Illumina sequencers (FIGS. 1A and 2). These images are transformed by an arbitrary translation, scaling, and rotation relative to the coordinate system used in the Illumina software. Alignment between the sequencing output and CHAMP images is further confounded by false-positive (e.g., spurious fluorescent signals) and false-negative cluster coordinates (e.g., fluorescent signals that are filtered out by the Illumina sequencing software). CHAMP overcomes this challenge by using alignment markers with known DNA sequences to match the spatial position of all fluorescent clusters to a corresponding record in the sequencing output file (FIG. 3A). A library consisting of the bacteriophage PhiX genome was used as the alignment marker because this DNA is included as an internal control and typically comprises 5-10% of all sequenced DNA clusters on every Illumina chip. This library also contains a unique sequencing adapter that can be selectively illuminated with a fluorescent primer (FIG. 2). Mapping the alignment markers and protein-bound clusters requires two stages: first, a rough alignment using Fourier-based cross correlation methods is performed, followed by a precision alignment using least-squares constellation mapping between FASTQ and de novo extracted clusters (see FIG. 3 and Star Methods). This is a specialized example of the image registration problem, and allows CHAMP to function with any fluorescence-based sequencing platform and TIRF microscope (see Discussion below).

d) Aligning Fluorescent Images and FASTQ Points: Overview

To identify the DNA sequence of each cluster, an image-processing pipeline was developed to process images collected by TIRF microscopy. To decode each cluster's sequence, its position was correlated to the corresponding record in the FASTQ file generated at the end of each MiSeq run. For each identified cluster, the FASTQ file reports the specifying lane, tile, and relative x-y coordinates. However, the FASTQ-supplied spatial information is reported in an arbitrary coordinate system that is scaled, rotated, and translated relative to the fluorescent images. An additional confounding factor is that FASTQ files do not report all fluorescent clusters (e.g., clusters that did not pass Illumina-specified quality control filters). In addition, some Illumina-reported clusters may also not light up in the fluorescent images. This can occur due to errors in the Illumina cluster identification pipeline, or possibly due to incomplete fluorescent labeling of the cluster during the experiments. As such, the mapping problem required finding the rotation, scale, x-offset, y-offset, and chip surface (both surfaces are imaged in a MiSeq chip) which best aligned the FASTQ points and imaged clusters. This was accomplished through two alignment stages: rough alignment and precision alignment, discussed below.

For the purposes of internal calibration. Illumina requires a percentage of each MiSeq run, typically 5-10% of all clusters, to be DNA from the small, thoroughly characterized phiX bacteriophage genome. Separate adapter chemistry is used for this phiX library, which can be accurately and specifically illuminated on any chip using complementary oligonucleotides. The phiX clusters do not contain a run-specific index barcode and are thus not demultiplexed as normal reads, but can be determined by mapping reads to the phiX genome. These phiX clusters provide a convenient resource for a variety of purposes, including alignment, categorization and intensity training, and as a control. The phiX clusters were illuminated by hybridizing them to a dye-conjugated oligo (Atto647-PCP or Cy3-PCP) during cluster re-generation and used the resulting fluorescent signals to align the fluorescent images with the corresponding FASTQ records.

(1) Stage 1: Rough Alignment

The rough alignment was performed through cross-correlation of FASTQ points and images using fast Fourier methods. Briefly, each FASTQ tile was converted to an image, each cluster represented as a radially symmetric Gaussian with σ of 0.25 μm, a typical cluster size. Cross-correlation was then performed via the formula

Cross correlation=| [symbol] ⁻¹[( [symbol] F)*· [symbol] T]|

with zero-padding enough to accommodate any offset, where [symbol] and [symbol] ⁻¹ are the fast forward and inverse 2D Fourier transforms, * is the complex conjugate, F is the FASTQ image, and T is the TIRF image. This allowed consideration of all x-y offsets (translation) in a computationally efficient manner, though did not inherently consider rotation or scale. For each TIRF image, the maximum cross-correlation was first found against two FASTQ tiles known from their position to not overlap the TIRF image in order to measure background noise level, after which correlations above a signal-to-noise cutoff of choice, 1.4 in the current work, indicated a good alignment. In order to achieve the first alignment, the parameter space around initial estimates of rotation, scale, and parity were exhaustively sampled. The first rough alignment established the approximate rotation and scale, and was performed on each MiSeq chip to account for small deviations in their mounting within the custom-built stage adapter. With reasonable estimates for these parameters, the Fourier-based alignment can be performed within 45 seconds on a desktop computer.

(2) Stage 2: Precision Alignment

Following rough alignment in the alignment marker image channel, precision alignment was performed via constellation mapping in all channels. The algorithm aimed to maximize the number of matches between FASTQ points and fluorescent clusters, forming the same “constellation” in each space. The mapping parameters were then quickly determined using linear least squares fitting.

First, cluster location information was extracted from the TIRF images. Astronomy software Source Extractor was used to fit two-dimensional Gaussian functions to the fluorescent clusters. Next, the nearest neighbors of FASTQ points were found in imaged cluster space and vice-versa using kd-trees. Two points which were nearest neighbors of each other in both directions were termed a mutual hit. Due to accrued noise—missing data in FASTQ space, missing data in imaged cluster space, and imperfect Gaussian calling—mutual hits were not by themselves high-confidence mappings. Mutual hits were further subcategorized by the statuses of other nearby clusters. If cluster A and FASTQ point B were mutual hits and no other cluster X or FASTQ point Y consider A or B nearest neighbors, then the mutual hit was termed an exclusive hit. If there was another cluster X whose nearest neighbor was FASTQ point B, or another FASTQ point Y whose nearest neighbor was cluster A, then the status of hit AB was determined by the distance to the closest such X or Y. If the closest such X or Y was more than 1.25 microns away—the diameter of a typical cluster—AB was termed a good mutual hit; otherwise AB was called a bad mutual hit. Using exclusive hits and good mutual hits, linear least squares fitting was performed to determine the final alignment. The precision alignment process, including both constellation identification and least squares fitting, is typically performed within 2.5 seconds on a desktop computer.

(3) Calculating Cluster Intensity

Machine-learned linear weighting of pixels was used to calculate the fluorescent intensity of each cluster. (see FIG. 3) For training, an experiment with only phiX clusters illuminated was used and restricted the analysis to exclusive and good mutual hits. Seven by seven pixel squares were extracted around each of these FASTQ points and linearized into feature vectors. Linear Discriminant Analysis (LDA) was then used to find pixel weights that best capture the intensity of a given cluster and penalize the intensity of neighboring clusters. The positive weights were used to calculate raw cluster intensities. To correct for variation in laser intensities across fields of view, cluster intensities were normalized within each run. The mode of pixel intensities of each image was calculated, and the intensity calculations in each image were normalized by the mode of the given image divided by the median of all modes.

(4) Data Analysis

(a) Calculating the Apparent Dissociation Constant:

Calculation of the apparent K_{d} value was performed for each sequence via curve fitting to the Hill equation (without cooperativity):

$${I}_{obs} = \frac{{I}_{max} - {I}_{min}}{1 + \frac{{K}_{d}}{x}} + {I}_{min}$$

where Iₘᵢₙ is the background intensity, Iₘₐₓ is the intensity of a fully saturated cluster, and the concentration values x and cluster intensity values I_{obs} are derived from the concentration gradient experiment. Iₘₐᵢₙ is calculated as the median intensity of negative control clusters in the lowest concentration point. Iₘₐₓ is determined separately for each concentration to normalize small differences in fluorescence intensities across the entire flowcell and between concentrations. At higher concentrations. DNA sequences that are perfectly complementary to the crRNA-Cascade complex become saturated and can be used as a reference to normalize between concentrations. To this end, Iₘₐₓ is calculated in two steps, using only clusters of the perfect target sequence. First, the K_{d} and a temporary, constant Iₘₐₓ, call it I_{max,const}, are fit jointly on the perfect target sequence clusters using information from all concentrations. Second, for each concentrations where median I_{obs} is greater than 90% of the fit I_{max,const}, Iₘₐₓ is solved for from the above equation, using the observed median cluster intensity as I_{obs}. At all preceding concentrations, I_{max,const} is used. These values of Iₘᵢₙ and Iₘₐₓ are then used to fit K_{d} for all other sequences. Error bars indicate the standard deviation of bootstrap K_{d} values.

(b) Position-Transition Model

The position transition model for change in apparent binding affinity (ΔABA) can be written as:

$$Δ ABA = {\sum }_{i = 1}^{35} {p}_{i} + ( {r}_{i} , {s}_{i} )$$

where pᵢ is the penalty, rᵢ is the reference base, and sᵢ is the sequenced base in the i^{th} position, and t(x, y) is the position-independent transition weight from x to y. The summation is carried out over all 35 positions in the minimal three-nucleotide PAM and the protospacer.

For computational efficiency, this in matrix form was cast. Each sequence was represented as a 35-by-12 indicator matrix S with rows representing each sequence position and columns representing each non-identity transition. The position penalties and transition weights were represented as vectors p and t. Then the above is written as

ΔABA=S:(p⊗t)

where : is the Frobenius inner product and ⊗ is the outer product. This was linearized and concatenated into multiple-sequence sparse matrices and fit using non-linear least squares. Having multiple reference sequences and normalizing the transition vector to have mean value one, obviated model degeneracy.

(c) Cas3 Penalties

The line of stoichiometric Cascade/Cas3 intensity was fit to all single-mismatch data with a mismatch in the fourth target position or greater. Cas3 penalties were then calculated as the observed Cas3 average intensity minus the expected stoichiometric intensity given average Cascade intensity, such that points furthest from the line represented sequences with the greatest difference in Cas3 vs. Cascade occupancy. Error bars are the SEM of intensity values.

(i) Exome Dataset Analysis

Exome reads were first trimmed with Trimmomatic 0.32 to remove Illumina adapter sequences. Trimmed reads were then mapped to the human genome using Bowtie2 2.2.3. The reads were then filtered for read quality and mapping phred score above 20, resulting in seven million high quality mapped reads, or an average 11-fold coverage in regions of interest. For each position with at least five overlapping imaged reads, intensity information from all reads was used to measure ABA, following the same procedure as with the synthetic libraries. This results in a flat signal across most of the genes, with peaks at off-target sites with high ABAs. The peak width reflects both the distribution of read lengths and coverage depth across the library. Below, this results was demonstrated in a triangle-shaped function.

Let randomly sheared DNA fragment R be the randomly placed genomic interval of length |R|, and consider ABA measurement site x and a nearby high-affinity binding site x_{b}. Then, the conditional probability that x is in R given x is in R decreases linearly from one to zero as |x−x_{b}| increases from zero to |R|. Letting read length be random, this gives

$$P ( {x}_{b} ∈ R \vert x ∈ R ) = {\sum }_{r = \vert x - {x}_{b} \vert}^{max \{ \vert R \vert \}} P ( \vert R \vert = r ) [ 1 - \frac{\vert x - {x}_{b} \vert}{r} ]$$

For |x−x_{b}| less than the minimum read length, this can be interpreted as an expectation, which simplifies to a perfectly triangular peak:

$$P ( {x}_{b} ∈ R \vert x ∈ R ) = 1 - 〈 \frac{1}{\vert R \vert} 〉 \vert x - {x}_{b} \vert$$

For the observed read length distribution, this is approximately true for |x−x_{b}|<100 bp (FIG. 7A). This accounts for the top >60% of the peak, so the theoretical peak shape is approximately triangular (FIG. 7B). When all reads have the same length, this results in a perfectly triangular peak. Due to library size-selection, read lengths were relatively focused around the mean length (FIG. 7A), so the resulting theoretical peak shape is approximately triangular (FIG. 7B). Using the observed read length distribution results in theoretical peaks with a full width at half maximum (FWHM) of 162 bp. The experimental peak shape was determined by summing the normalized peak shapes from the top thirty high-affinity DNA binding sites. Remarkably, this result is in near quantitative agreement with the theoretical calculations with an observed FWHM of 210 bp. Deviation from the theoretical shape is due to finite coverage, bias in shearing sites, and the non-linear map from reads included to measured ABA. The more conservative estimate of 210 bp was therefore used as the cutoff for determining the underlying consensus motif. This motif was determined by searching a 210 bp window around the peak of the ABA curves for the presence of a high-affinity PAM and crRNA-complementary DNA. The results were plotted as a logo using WebLogo.

(ii) Data and Software Availability

The source code for cluster identification, spatial registration, and binding affinity calculations is available via GitHub.

### D. References

Amitai, G., and Sorek, R. (2016). CRISPR-Cas adaptation: insights into the mechanism of action. Nat. Rev. Microbiol. 14, 67-76.
Berger, M. F., Philippakis, A. A., Qureshi, A. M., He, F. S., Estep, P. W., and Bulyk, M. L. (2006). Compact, universal DNA microarrays to comprehensively determine transcription-factor binding site specificities. Nat. Biotechnol. 24, 1429-1435.
Bertin, E., and Arnouts, S. (1996). SExtractor: Software for source extraction. Astron. Astrophys. Suppl. Ser. 117, 12.
Blosser, T. R., Loeff, L., Westra, E. R., Vlot, M., Künne, T., Sobota, M., Dekker, C., Brouns, S. J. J., and Joo, C. (2015). Two distinct DNA binding modes guide dual roles of a CRISPR-Cas protein complex. Mol. Cell 58, 60-70.
Bolger, A. M., Lohse, M., and Usadel, B. (2014). Trimmomatic: a flexible trimmer for Illumina sequence data. Bioinforma. Oxf. Engl. 30, 2114-2120.
Bolukbasi, M. F., Gupta, A., and Wolfe, S. A. (2016). Creating and evaluating accurate CRISPR-Cas9 scalpels for genomic surgery. Nat. Methods 13, 41-50.
Buenrostro, J. D., Araya, C. L., Chircus, L. M., Layton, C. J., Chang, H. Y., Snyder, M. P., and Greenleaf, W. J. (2014). Quantitative analysis of RNA-protein interactions on a massively parallel array reveals biophysical and evolutionary landscapes. Nat. Biotechnol. 32, 562-568.
Caliando, B. J., and Voigt, C. A. (2015). Targeted DNA degradation using a CRISPR device stably carried in the host genome. Nat. Commun. 6, 6989.
Carlson, C. D., Warren, C. L., Hauschild, K. E., Ozers, M. S., Qadir, N., Bhimsaria, D., Lee, Y., Cerrina, F., and Ansari, A. Z. (2010). Specificity landscapes of DNA binding molecules elucidate biological function. Proc. Natl. Acad. Sci. 107, 4544-4549.
Crooks, G. E., Hon, G., Chandonia, J.-M., and Brenner, S. E. (2004). WebLogo: a sequence logo generator. Genome Res. 14, 1188-1190.
Edelstein, A. D., Tsuchida, M. A., Amodaj, N., Pinkard, H., Vale, R. D., and Stuurman, N. (2014). Advanced methods of microscope control using Manager software. J. Biol. Methods 1, 10.
Efron, B., and Tibshirani, R. J. (1993). An Introduction to the Bootstrap (New York: Chapman and Hall/CRC).
Fineran, P. C., Gerritzen, M. J. H., Suárez-Diez, M., Künne, T., Boekhorst, J., van Hijum, S. A. F. T., Staals, R. H. J., and Brouns, S. J. J. (2014). Degenerate target sites mediate rapid primed CRISPR adaptation. Proc. Natl. Acad. Sci. U.S.A. 111, E1629-1638.
Hayes. R. P., Xiao, Y., Ding, F., van Erp, P. B. G., Rajashankar, K., Bailey, S., Wiedenheft, B., and Ke, A. (2016). Structural basis for promiscuous PAM recognition in type I-E Cascade from E. coli. Nature advance online publication.
Heler, R., Samai, P., Modell, J. W., Weiner, C., Goldberg, G. W., Bikard, D., and Marraffini. L. A. (2015). Cas9 specifies functional viral targets during CRISPR-Cas adaptation. Nature 519, 199-202.
Homola, J. (2008). Surface plasmon resonance sensors for detection of chemical and biological species. Chem. Rev. 108, 462-493.
Horvath, P., Romero, D. A., Coûté-Monvoisin, A.-C., Richards, M., Deveau, H., Moineau, S., Boyaval, P., Fremaux, C., and Barrangou, R. (2008). Diversity, Activity, and Evolution of CRISPR Loci in Streptococcus thermophilus. J. Bacteriol. 190, 1401-1412.
Hsu, P. D., Lander, E. S., and Zhang, F. (2014). Development and Applications of CRISPR-Cas9 for Genome Engineering. Cell 157, 1262-1278.
Hsu, P. D., Scott. D. A., Weinstein, J. A., Ran, F. A., Konermann, S., Agarwala, V., Li, Y., Fine, E. J., Wu, X., Shalem, O., et al. (2013). DNA targeting specificity of RNA-guided Cas9 nucleases. Nat. Biotechnol. 31, 827-832.
Huo, Y., Nam, K. H., Ding, F., Lee, H., Wu, L., Xiao, Y., Farchione Jr, M. D., Zhou, S., Rajashankar, K., Kurinov, I., et al. (2014). Structures of CRISPR Cas3 offer mechanistic insights into Cascade-activated DNA unwinding and degradation. Nat. Struct. Mol. Biol. 21, 771-777.
Jackson, R. N., Golden, S. M., Erp, P. B. G. van, Carter, J., Westra, E. R., Brouns, S. J. J., Oost, J. van der, Terwilliger, T. C., Read, R. J., and Wiedenheft, B. (2014). Crystal structure of the CRISPR RNA-guided surveillance complex from Escherichia coli. Science 345, 1473-1479.
Jiang, F., Zhou, K., Ma, L., Gressel, S., and Doudna, J. A. (2015). A Cas9-guide RNA complex preorganized for target DNA recognition. Science 348, 1477-1481.
Johnson, D. S., Mortazavi, A., Myers, R. M., and Wold, B. (2007). Genome-wide mapping of in vivo protein-DNA interactions. Science 316, 1497-1502.
Johna, A., Kivioja, T., Toivonen, J., Cheng, L., Wei, G., Enge, M., Taipale, M. Vaquerizas, J. M., Yan, J., Sillanpää, M. J., et al. (2010). Multiplexed massively parallel SELEX for characterization of human transcription factor binding specificities. Genome Res. 20, 861-873.
Jore, M. M., Lundgren, M., van Duijn, E., Bultema, J. B., Westra. E. R., Waghmare, S. P., Wiedenheft, B., Pul, Ü., Wurm, R., Wagner, R., et al. (2011). Structural basis for CRISPR RNA-guided DNA recognition by Cascade. Nat. Struct. Mol. Biol. 18, 529-536.
Kim, D., Bae, S., Park, J., Kim, E., Kim, S., Yu, H. R., Hwang, J., Kim, J.-I., and Kim, J.-S. (2015). Digenome-seq: genome-wide profiling of CRISPR-Cas9 off-target effects in human cells. Nat. Methods 12, 237-243, 1 p following 243.
Lambert, N., Robertson, A., Jangi, M., McGeary, S., Sharp, P. A., and Burge, C. B. (2014). RNA Bind-n-Seq: Quantitative Assessment of the Sequence and Structural Binding Specificity of RNA Binding Proteins. Mol. Cell 54, 887-900.
Langmead, B., and Salzberg, S. L. (2012). Fast gapped-read alignment with Bowtie 2. Nat. Methods 9, 357-359.
Leenay, R. T., Maksimchuk, K. R., Slotkowski, R. A., Agrawal, R. N., Gomaa, A. A., Briner, A. E., Barrangou, R., and Beisel, C. L. (2016). Identifying and Visualizing Functional PAM Diversity across CRISPR-Cas Systems. Mol. Cell 62, 137-147.
Luo, M. L., Mullis, A. S., Leenay, R. T., and Beisel, C. L. (2014). Repurposing endogenous type I CRISPR-Cas systems for programmable gene repression. Nucleic Acids Res. gku971.
Makarova, K. S., Wolf, Y. I., Alkhnbashi, O. S., Costa, F., Shah, S. A., Saunders, S. J., Barrangou, R., Brouns, S. J. J., Charpentier, E., Haft, D. H., et al. (2015). An updated evolutionary classification of CRISPR-Cas systems. Nat. Rev. Microbiol. 13, 722-736.
Maneewongvatana, S., and Mount, D. M. (1999). It's okay to be skinny, if your friends are fat. In Center for Geometric Computing 4th Annual Workshop on Computational Geometry, pp. 1-8.
Marraffini, L. A. (2015). CRISPR-Cas immunity in prokaryotes. Nature 526, 55-61.
Marraffini, L. A., and Sontheimer, E. J. (2010). CRISPR interference: RNA-directed adaptive immunity in bacteria and archaea. Nat. Rev. Genet. 11, 181-190.
Nutiu, R., Friedman, R. C., Luo. S., Khrebtukova, I., Silva, D., Li, R., Zhang, L., Schroth, G. P., and Burge, C. B. (2011). Direct measurement of DNA affinity landscapes on a high-throughput sequencing instrument. Nat. Biotechnol. 29, 659-664.
O'Geen, H., Henry, I. M., Bhakta, M. S., Meckler, J. F., and Segal, D. J. (2015). A genome-wide analysis of Cas9 binding specificity using ChIP-seq and targeted sequence capture. Nucleic Acids Res. gkv137.
Ondov, B. D., Bergman, N. H., and Phillippy, A. M. (2011). Interactive metagenomics visualization in a Web browser. BMC Bioinformatics 12, 385.
Press, W. H. (2007). Numerical Recipes 3rd Edition: The Art of Scientific Computing (Cambridge, UK; New York: Cambridge University Press).
Qavi, A. J., Washburn, A. L., Byeon, J.-Y., and Bailey, R. C. (2009). Label-free technologies for quantitative multiparameter biological analysis. Anal. Bioanal. Chem. 394, 121-135.
Ran, F. A., Cong, L., Yan, W. X., Scott, D. A., Gootenherg, J. S., Kriz, A. J., Zetsche, B., Shalem, O., Wu. X., Makarova, K. S., et al. (2015). In vivo genome editing using Staphylococcus aureus Cas9. Nature 520, 186-191.
Redding, S., Sternberg, S. H., Marshall, M., Gibb, B., Bhat, P., Guegler, C. K., Wiedenheft, B., Doudna, J. A., and Greene, E. C. (2015). Surveillance and Processing of Foreign DNA by the Escherichia coli CRISPR-Cas System. Cell 163, 854-865.
Rutkauskas, M., Sinkunas, T., Songailiene, I., Tikhomirova, M. S., Siksnys, V., and Seidel, R. (2015). Directional R-Loop Formation by the CRISPR-Cas Surveillance Complex Cascade Provides Efficient Off-Target Site Rejection. Cell Rep. 10, 1534-1543.
Sander, J. D., and Joung, J. K. (2014). CRISPR-Cas systems for editing, regulating and targeting genomes. Nat. Biotechnol. 32, 347-355.
Sashital, D. G., Wiedenheft, B., and Doudna, J. A. (2012). Mechanism of Foreign DNA Selection in a Bacterial Adaptive Immune System. Mol. Cell 46, 606-615.
Schirle, N. T., and MacRae, I. J. (2012). The crystal structure of human Argonaute2. Science 336, 1037-1040.
Semenova, E., Jore, M. M., Datsenko, K. A., Semenova, A., Westra, E. R., Wanner, B., Oost, J. van der, Brouns, S. J. J., and Severinov, K. (2011). Interference by clustered regularly interspaced short palindromic repeat (CRISPR) RNA is governed by a seed sequence. Proc. Natl. Acad. Sci. 108, 10098-10103.
Semenova, E., Savitskaya. E., Musharova, O., Strotskaya, A., Vorontsova, D., Datsenko, K. A., Logacheva, M. D., and Severinov, K. (2016). Highly efficient primed spacer acquisition from targets destroyed by the Escherichia coli type I-E CRISPR-Cas interfering complex. Proc. Natl. Acad. Sci. 113, 7626-7631.
Shumaker-Parry, J. S., Aebersold, R., and Campbell, C. T. (2004). Parallel, quantitative measurement of protein binding to a 120-element double-stranded DNA array in real time using surface plasmon resonance microscopy. Anal. Chem. 76, 2071-2082.
Sorek, R., Lawrence, C. M., and Wiedenheft, B. (2013). CRISPR-Mediated Adaptive Immune Systems in Bacteria and Archaea. Annu. Rev. Biochem. 82, 237-266.
Staals, R. H. J., Jackson, S. A., Biswas, A., Brouns, S. J. J., Brown, C. M., and Fineran, P. C. (2016). Interference-driven spacer acquisition is dominant over naive and primed adaptation in a native CRISPR-Cas system. Nat. Commun. 7, 12853.
Stormo, G. D., and Zhao, Y. (2010). Determining the specificity of protein-DNA interactions. Nat. Rev. Genet. 11, 751-760.
Stormo, G. D., Zuo, Z., and Chang, Y. K. (2015). Spec-seq: determining protein-DNA binding specificity by sequencing. Brief. Funct. Genomics 14, 30-38.
Szczelkun, M. D., Tikhomirova, M. S., Sinkunas. T., Gasiunas, G., Karvelis, T., Pschera, P., Siksnys, V., and Seidel, R. (2014). Direct observation of R-loop formation by single RNA-guided Cas9 and Cascade effector complexes. Proc. Natl. Acad. Sci. 11, 9798-9803.
Tome, J. M., Ozer, A., Pagano, J. M., Gheba, D., Schroth, G. P., and Lis, J. T. (2014). Comprehensive analysis of RNA-protein interactions by high-throughput sequencing-RNA affinity profiling. Nat. Methods 11, 683-688.
van Erp, P. B. G., Jackson, R. N., Carter, J., Golden, S. M., Bailey, S., and Wiedenheft, B. (2015). Mechanism of CRISPR-RNA guided recognition of DNA targets in Escherichia coli. Nucleic Acids Res. 43, 8381-8391.
Wiedenheft, B., van Duijn. E., Bultema. J. B., Bultema, J., Waghmare, S. P., Waghmare, S., Zhou, K., Barendregt, A., Westphal, W., Heck, A. J. R., et al. (2011). RNA-guided complex from a bacterial immune system enhances target recognition through seed sequence interactions. Proc. Natl. Acad. Sci. U.S.A. 108, 10092-10097.
Wright, A. V., Nuñez, J. K., and Doudna, J. A. (2016). Biology and Applications of CRISPR Systems: Harnessing Nature's Toolbox for Genome Engineering. Cell 164, 29-44.
Wu, X., Kriz, A. J., and Sharp. P. A. (2014). Target specificity of the CRISPR-Cas9 system. Quant. Biol. 2, 59-70.
Xue, C., Seetharam, A. S., Musharova, O., Severinov, K., J. Brouns, S. J., Severin, A. J., and Sashital, D. G. (2015). CRISPR interference and priming varies with individual spacer sequences. Nucleic Acids Res. 43, 10831-10847.
Xue, C., Whitis, N. R., and Sashital, D. G. (2016). Conformational Control of Cascade Interference and Priming Activities in CRISPR Immunity. Mol. Cell 64, 826-834.
Zhao, H., Sheng, G., Wang, J., Wang. M., Bunkoczi. G., Gong. W., Wei, Z., and Wang, Y. (2014). Crystal structure of the RNA-guided immune surveillance Cascade complex in Escherichia coli. Nature 515, 147-150.
Zitová, B., and Flusser, J. (2003). Image registration methods: a survey. Image Vis. Comput. 21, 977-1000.
Zykovich, A., Korf, I., and Segal, D. J. (2009). Bind-n-Seq: high-throughput analysis of in vitro protein-DNA interactions using massively parallel sequencing. Nucleic Acids Res. 37, e151-e151.

1. A method for determining protein-nucleic acid interactions, the method comprising: exposing nucleic acid clusters on a high-throughput array to one or more fluorescently labeled proteins; and detecting protein-nucleic acid interactions by fluorescent imaging.

2. (canceled)

3. (canceled)

4. (canceled)

5. (canceled)

6. The method of claim 1, wherein the high throughput array is a next-generation sequencing (NGS) array.

7. The method of claim 1, wherein the high throughput array is a microarray.

8. The method of claim 1, wherein the high throughput array is an Illumina® chip.

9. The method of claim 1, wherein the high throughput array has previously been used for sequencing nucleic acids.

10. The method of claim 1, wherein the high throughput array comprises 1 million or more unique nucleic acid clusters.

11. The method of claim 1, wherein a fluorescent microscope is used to image protein-nucleic acid interactions.

12. The method of claim 11, wherein multi-color co-localization is used to determine protein-nucleic acid interaction.

13. The method of claim 11, wherein time-dependent kinetics of protein-nucleic acid interactions are measured.

14. The method of claim 11, wherein fluorescent resonant energy transfer (FRET) is used to determine protein-nucleic acid interaction.

15. The method of claim 11, wherein the microscope is a total internal reflection fluorescence (TIRF) microscope.

16. The method of claim 1, further comprising using a subset of nucleic acid clusters as alignment markers to align spatial information obtained via sequencing with fluorescent imaging data obtained to determine specific protein-nucleic acid interactions.

17. The method of claim 16, wherein fluorescent oligonucleotide primers are hybridized to the subset of the DNA clusters and used as alignment markers.

18. A chip hybridized association-mapping platform for determining protein-nucleic acid interaction, the platform comprising nucleic acid clusters on a high-throughput array and one or more fluorescently labeled proteins.

19. (canceled)

20. (canceled)

21. (canceled)

22. (canceled)

23. The platform of claim 18, wherein the high throughput array is a next-generation sequencing (NGS) array.

24. The platform of claim 18, wherein the high throughput array is a microarray.

25. The platform of claim 18, wherein the high throughput array is an Illumina® chip.

26. The platform of claim 18, wherein the high throughput array has previously been used for sequencing nucleic acids.

27. The platform of claim 18, wherein the high throughput array comprises 1 million or more unique nucleic acid clusters.

28. The platform of claim 18, wherein the platform further comprises a fluorescent microscope.

29. The platform of claim 28, wherein the microscope is a total internal reflection fluorescence (TIRF) microscope.

30. The platform of claim 18, further comprising fluorescent oligonucleotide primers used as alignment markers.


SEQUENCE LISTING (11 sequences)

SEQ ID NO: 1 - DNA, 177 bp, Artificial Sequence
  Synthetic Construct
aatgatacggcgaccaccgagatctacactctttccctacacgacgctcttccgatctaaggccgaattctcaccggccccaaggtattcaagagatcggaagagcacacgtctgaactccagtcacttgttcttttgcactaccgtcaggtaatctcgtatgccgtcttctgcttg

SEQ ID NO: 2 - DNA, 177 bp, Artificial Sequence
  Synthetic Construct
aatgatacggcgaccaccgagatctacactctttccctacacgacgctcttccgatctaaggccgatttctctccggctccaagttattctagagatcggaagagcacacgtctgaactccagtcacttgttcttttgcactaccgtcaggtaatctcgtatgccgtcttctgcttg

SEQ ID NO: 3 - DNA, 177 bp, Artificial Sequence
  Synthetic Construct
aatgatacggcgaccaccgagatctacactctttccctacacgacgctcttccgatctaaggccgacttctccccggccccaagctattccagagatcggaagagcacacgtctgaactccagtcacttgttcttttgcactaccgtcaggtaatctcgtatgccgtcttctgcttg

SEQ ID NO: 4 - DNA, 177 bp, Artificial Sequence
  Synthetic Construct
  misc_feature (62)..(68): n is a, c, g, or t
aatgatacggcgaccaccgagatctacactctttccctacacgacgctcttccgatctaagnnnnnnntctcaccggccccaaggtattcaagagatcggaagagcacacgtctgaactccagtcacttgttcttttgcactaccgtcaggtaatctcgtatgccgtcttctgcttg

SEQ ID NO: 5 - DNA, 177 bp, Artificial Sequence; identical to SEQ ID NO: 1
  Synthetic Construct

SEQ ID NO: 6 - DNA, 177 bp, Artificial Sequence
  Synthetic Construct
aatgatacggcgaccaccgagatctacactctttccctacacgacgctcttccgatctaagccagtgataagtggaatgccatgtgggctgtcagatcggaagagcacacgtctgaactccagtcacttgttcttttgcactaccgtcaggtaatctcgtatgccgtcttctgcttg

SEQ ID NO: 7 - DNA, 21 bp, Artificial Sequence
  Synthetic Construct
gtgactggagttcagacgtgt

SEQ ID NO: 8 - DNA, 24 bp, Artificial Sequence
  Synthetic Construct
cggtctcggcattcctgctgaacc

SEQ ID NO: 9 - DNA, 24 bp, Artificial Sequence
  Synthetic Construct
cggtctcggcattcctgctgaacc

SEQ ID NO: 10 - DNA, 22 bp, Artificial Sequence
  Synthetic Construct
aatgatacggcgaccaccgaga

SEQ ID NO: 11 - DNA, 22 bp, Artificial Sequence
  Synthetic Construct
aatgatacggcgaccaccgaga